SEQUENCE LISTING (6 sequences)

SEQ ID NO: 1 - DNA, 2076 bp, Homo sapiens
  CDS (431)..(1690)
cggggaaggggagggaggagggggacgagggctctggcgggtttggaggggctgaacatcgcggggtgttctggtgtcccccgccccgcctctccaaaaagctacaccgacgcggaccgcggcggcgtcctccctcgccctcgcttcacctcgcgggctccgaatgcggggagctcggatgtccggtttcctgtgaggcttttacctgacacccgccgcctttccccggcactggctgggagggcgccctgcaaagttgggaacgcggagccccggacccgctcccgccgcctccggctcgcccagggggggtcgccgggaggagcccgggggagagggaccaggaggggcccgcggcctcgcaggggcgcccgcgcccccacccctgcccccgccagcggaccggtcccccacccccggtccttccaccatgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaagattgtactgttttccagttcatcgattttctattatggaaaactgtgttgccacagtagaactgtctgtgaacagagagacccttgtgggtccatgctaacaaagacaaaagtctgtctttcctgaaccatgtggataactttacagaaatggactggagctcatctgcaaaaggcctcttgtaaagactggttttctgccaatgaccaaacagccaagattttcctcttgtgatttctttaaaagaatgactatataatttatttccactaaaaatattgtttctgcattcatttttatagcaacaacaattggtaaaactcactgtgatcaatatttttatatcatgcaaaatatgtttaaaataaaatgaaaattgtattat

SEQ ID NO: 2 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 3 - DNA, 4450 bp, Homo sapiens
  misc_feature (22)..(2346): extracellular domain
acccacgcgcagcggccggagatgcagcggggcgccgcgctgtgcctgcgactgtggctctgcctgggactcctggacggcctggtgagtgactactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctctatgacatccagctgttgcccaggaagtcgctggagctgctggtaggggagaagctggtcctcaactgcaccgtgtgggctgagtttaactcaggtgtcacctttgactgggactacccagggaagcaggcagagcggggtaagtgggtgcccgagcgacgctcccaacagacccacacagaactctccagcatcctgaccatccacaacgtcagccagcacgacctgggctcgtatgtgtgcaaggccaacaacggcatccagcgatttcgggagagcaccgaggtcattgtgcatgaaaatcccttcatcagcgtcgagtggctcaaaggacccatcctggaggccacggcaggagacgagctggtgaagctgcccgtgaagctggcagcgtaccccccgcccgagttccagtggtacaaggatggaaaggcactgtccgggcgccacagtccacatgccctggtgctcaaggaggtgacagaggccagcacaggcacctacaccctcgccctgtggaactccgctgctggcctgaggcgcaacatcagcctggagctggtggtgaatgtgcccccccagatacatgagaaggaggcctcctcccccagcatctactcgcgtcacagccgccaggccctcacctgcacggcctacggggtgcccctgcctctcagcatccagtggcactggcggccctggacaccctgcaagatgtttgcccagcgtagtctccggcggcggcagcagcaagacctcatgccacagtgccgtgactggagggcggtgaccacgcaggatgccgtgaaccccatcgagagcctggacacctggaccgagtttgtggagggaaagaataagactgtgagcaagctggtgatccagaatgccaacgtgtctgccatgtacaagtgtgtggtctccaacaaggtgggccaggatgagcggctcatctacttctatgtgaccaccatccccgacggcttcaccatcgaatccaagccatccgaggagctactagagggccagccggtgctcctgagctgccaagccgacagctacaagtacgagcatctgcgctggtaccgcctcaacctgtccacgctgcacgatgcgcacgggaacccgcttctgctcgactgcaagaacgtgcatctgttcgccacccctctggccgccagcctggaggaggtggcacctggggcgcgccacgccacgctcagcctgagtatcccccgcgtcgcgcccgagcacgagggccactatgtgtgcgaagtgcaagaccggcgcagccatgacaagcactgccacaagaagtacctgtcggtgcaggccctggaagcccctcggctcacgcagaacttgaccgacctcctggtgaacgtgagcgactcgctggagatgcagtgcttggtggccggagcgcacgcgcccagcatcgtgtggtacaaagacgagaggctgctggaggaaaagtctggagtcgacttggcggactccaaccagaagctgagcatccagcgcgtgcgcgaggaggatgcgggaccgtatctgtgcagcgtgtgcagacccaagggctgcgtcaactcctccgccagcgtggccgtggaaggctccgaggataagggcagcatggagatcgtgatccttgtcggtaccggcgtcatcgctgtcttcttctgggtcctcctcctcctcatcttctgtaacatgaggaggccggcccacgcagacatcaagacgggctacctgtccatcatcatggaccccggggaggtgcctctggaggagcaatgcgaatacctgtcctacgatgccagccagtgggaattcccccgagagcggctgcacctggggagagtgctcggctacggcgccttcgggaaggtggtggaagcctccgctttcggcatccacaagggcagcagctgtgacaccgtggccgtgaaaatgctgaaagagggcgccacggccagcgagcagcgcgcgctgatgtcggagctcaagatcctcattcacatcggcaaccacctcaacgtggtcaacctcctcggggcgtgcaccaagccgcagggccccctcatggtgatcgtggagttctgcaagtacggcaacctctccaacttcctgcgcgccaagcgggacgccttcagcccctgcgcggagaagtctcccgagcagcgcggacgcttccgcgccatggtggagctcgccaggctggatcggaggcggccggggagcagcgacagggtcctcttcgcgcggttctcgaagaccgagggcggagcgaggcgggcttctccagaccaagaagctgaggacctgtggctgagcccgctgaccatggaagatcttgtctgctacagcttccaggtggccagagggatggagttcctggcttcccgaaagtgcatccacagagacctggctgctcggaacattctgctgtcggaaagcgacgtggtgaagatctgtgactttggccttgcccgggacatctacaaagaccccgactacgtccgcaagggcagtgcccggctgcccctgaagtggatggcccctgaaagcatcttcgacaaggtgtacaccacgcagagtgacgtgtggtcctttggggtgcttctctgggagatcttctctctgggggcctccccgtaccctggggtgcagatcaatgaggagttctgccagcgcgtgagagacggcacaaggatgagggccccggagctggccactcccgccatacgccacatcatgctgaactgctggtccggagaccccaaggcgagacctgcattctcggacctggtggagatcctgggggacctgctccagggcaggggcctgcaagaggaagaggaggtctgcatggccccgcgcagctctcagagctcagaagagggcagcttctcgcaggtgtccaccatggccctacacatcgcccaggctgacgctgaggacagcccgccaagcctgcagcgccacagcctggccgccaggtattacaactgggtgtcctttcccgggtgcctggccagaggggctgagacccgtggttcctccaggatgaagacatttgaggaattccccatgaccccaacgacctacaaaggctctgtggacaaccagacagacagtgggatggtgctggcctcggaggagtttgagcagatagagagcaggcatagacaagaaagcggcttcaggtagctgaagcagagagagagaaggcagcatacgtcagcattttcttctctgcacttataagaaagatcaaagactttaagactttcgctatttcttctactgctatctactacaaacttcaaagaggaaccaggaggacaagaggagcatgaaagtggacaaggagtgtgaccactgaagcaccacagggaggggttaggcctccggatgactgcgggcaggcctggataatatccagcctcccacaagaagctggtggagcagagtgttccctgactcctccaaggaaagggagacgccctttcatggtctgctgagtaacaggtgccttcccagacactggcgttactgcttgaccaaagagccctcaagcggcccttatgccagcgtgacagagggctcacctcttgccttctaggtcacttctcacaatgtcccttcagcacctgaccctgtgcccgccgattattccttggtaatatgagtaatacatcaaagagtagtattaaaagctaattaatcatgtttataaaaa

SEQ ID NO: 4 - protein, 1298 aa, Homo sapiens
MQRGAALCLRLWLCLGLLDGLVSDYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGPYLCSVCRPKGCVNSSASVAVEGSEDKGSMEIVILVGTGVIAVFFWVLLLLIFCNMRRPAHADIKTGYLSIIMDPGEVPLEEQCEYLSYDASQWEFPRERLHLGRVLGYGAFGKVVEASAFGIHKGSSCDTVAVKMLKEGATASEQRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRVRDGTRMRAPELATPAIRHIMLNCWSGDPKARPAFSDLVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFR

SEQ ID NO: 5 - DNA, 25 bp, Artificial sequence
  Synthetic primer
tacaaagcttttcgccaccatgcag

SEQ ID NO: 6 - DNA, 27 bp, Artificial sequence
  Synthetic primer
tacaggatcctcatgcacaatgacctc

(19) United States
(12) Patent Application Publication
(10) Pub. No.: US 2004/0120950 A1
(43) Pub. Date: Jun. 24, 2004

(54) Modulation of VEGF-C/VEGFR-3 interactions in the treatment of rheumatoid arthritis

(76) Inventors: Kari Alitalo, Helsinki (FI); Karri Paavonen, Helsinki (FI); Yrjo Konttinen, Helsinki (FI)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/326,048

(22) Filed: Dec. 20, 2002

(51) Int. Cl. A61K 39/395; G01N 33/53; G01N 33/567

(52) U.S. Cl. 424/145.1; 435/7.2

(57) ABSTRACT

The present invention relates to methods for treating an individual exhibiting symptoms of chronic arthridites, as identified by an elevated level of VEGF-C expression at synovial sites, and provides materials and methods for the modulation of VEGF-C/VEGFR-3 ligand-receptor interactions as a treatment for chronic arthridites.

### FIELD OF THE INVENTION

The present invention provides materials and methods for the modulation of VEGF-C/VEGFR-3 ligand-receptor interactions as a treatment for chronic arthridites.

### BACKGROUND OF THE INVENTION

Angiogenesis, the formation of new blood vessels from preexisting ones, is essential in physiological processes such as inflammation and wound healing, embryonic development, tissue and organ regeneration and during the female reproductive cycle (Folkman J. Nat. Med. 1:27-31, 1995). Normally, a carefully maintained balance prevails between blood vessel growth stimulating factors such as vascular endothelial growth factor and blood vessel growth inhibitors such as endostatin and angiostatin (Folkman et al, Cell. 87:1153-5, 1996).

The physiology of the vascular system, embryonic vasculogenesis and angiogenesis, blood clotting, wound healing and reproduction, as well as several diseases, involve the vascular endothelium which line the blood vessels. The development of the vascular tree occurs through angiogenesis, and, according to prevailing theories, the formation of the lymphatic system starts shortly after arterial and venous development by sprouting from veins. See Sabin, F. R., Am. J. Anat., 9:43 (1909); and van der Putte, S. C. J, Adv. Anat. Embryol. Cell Biol., 51:3 (1975). After the fetal period, endothelial cells proliferate very slowly, except during angiogenesis associated with neovascularization. Growth factors stimulating angiogenesis exert their effects mainly via specific endothelial cell surface receptor tyrosine kinases.

A large family of vascular endothelial growth factors have been identified which, together with their receptors, play important roles in both vasculogenesis and angiogenesis [Risau et al., Dev Biol 125:441-450 (1988); Zachary, Intl J Biochem Cell Bio 30:1169-1174 (1998); Neufeld et al., FASEB J 13:9-22 (1999); Ferrara, J Mol Med 77:527-543 (1999)]. Both processes depend on tightly controlled endothelial cell proliferation, migration, differentiation, and survival.

The most-widely studied growth factor is Vascular Endothelial Growth Factor (VEGF), a member of the PDGF family of proteins. Vascular endothelial growth factor is a dimeric glycoprotein of disulfide-linked 23 kDa subunits, discovered because of its mitogenic activity toward endothelial cells and its ability to induce vessel permeability (hence its alternative name vascular permeability factor). Other reported effects of VEGF include the mobilization of intracellular Ca²⁺, the induction of plasminogen activator and plasminogen activator inhibitor-1 synthesis, stimulation of hexose transport in endothelial cells, and promotion of monocyte migration in vitro. Four VEGF isoforms, encoded by distinct mRNA splicing variants, appear to be equally capable of stimulating mitogenesis of endothelial cells. The 121 and 165 amino acid isoforms of VEGF are secreted in a soluble form, whereas the isoforms of 189 and 206 amino acid residues remain associated with the cell surface and have a strong affinity for heparin. Soluble non-heparin-binding and heparin-binding forms have also been described for the related placenta growth factor (P1GF; 131 and 152 amino acids, respectively), which is expressed in placenta, trophoblastic tumors, and cultured human endothelial cells.

A VEGF homologue, VEGF-C, was recently identified as a growth factor for the lymphatic vascular system. See International Patent Application No. PCT/US98/01973, published as WO 98/33917 on Aug. 6, 1998. One of its receptors, VEGFR-3 (Flt-4), is expressed in all endothelial cells during early embryogenesis. During later stages of development, the expression of VEGFR-3 becomes restricted to lymphatic vessels (Alitalo et al. U.S. Pat. Nos. 6,107,046 and 5,776,755; Joukov et al., EMBO J. 15:290-298, 1996; Aprelikova et al., Cancer Res. 52:746-748, 1992). It has been shown that VEGF-C stimulates lymphangiogenesis in vivo, and transgenic mice overexpressing VEGF-C in the skin are characterized by specific hyperplasia of the lymphatic network. Furthermore, VEGF-C has also been shown to induce angiogenesis in vitro and in vivo. As VEGFR-3 was also reported to be up-regulated on tumor blood vessels, it has been suggested that signaling of VEGF-C via VEGFR-3 may stimulate both tumor lymphangiogenesis and angiogenesis (International Patent Application No. PCT/US99/23525, published as WO 00/21560, incorporated herein by reference; Valtola et al., Am. J. Pathol. 154 1382-1390, 1999; Kubo et al., Blood 96, 546-553, 2000).

In addition to playing a key role in the progression of cancer, dysfunction of the endothelial cell regulatory system also is involved in several diseases associated with abnormal angiogenesis, such as proliferative retinopathies, age-related macular degeneration, rheumatoid arthritis, and psoriasis.

The occurrence of secreted blood vessel growth factors and growth inhibitors is unbalanced in rheumatoid arthritis (RA) and other chronic arthridites, which have a strong angiogenic component (Folkman J., 1995, supra). VEGF has been found to be a prime angiogenic molecule in RA (Afuwape et al., Histol Histopathol. 17:961-72, 2002; Paleolog et al., Angiogenesis 2:295-307, 1998), with VEGF and its receptors VEGFR-1 (Flt-1) and VEGFR-2 (KDR/Flk-1) detected in vascular endothelium in synovial membranes (Fava et al., J. Exp. Med. 180:341-46. 1994; Ikeda et al., J. Pathol. 191:426-33, 2000). Koch et al., in J. Immunol. 152: 4149, 1994, showed that the mitogenic activity of microvascular endothelial cells found in rheumatoid arthritis (RA) synovial tissue explants can be reduced by treatment with VEGF-specific antibodies and Ferrara et al, in U.S. Patent Application No. 20020032313 A1 suggest that hVEGF antagonists may be used to non-specifically inhibit VEGF interactions in several diseases found to involve neoangiogenesis, including RA.

Rheumatoid arthritis is thought to be mediated primarily by autoreactive immune cells migrating into synovial sites which results in localized joint swelling and inflammation. Several widely administered treatments for arthritis involve the use of nonspecific cytotoxic immunosuppressive drugs, e.g. methotrexate, cyclophosphamide, Imuran (azathioprine) and cyclosporin A. These drugs, as well as commonly used glucocorticoids (methylprednisolone, prednisone) suppress the entire immune system and are incapable of selectively suppressing the abnormal immune response. This global restraint of the immune system over time increases the risk of infection.

Other common treatments for rheumatoid arthritis and additional general arthritic conditions include NSAIDS such as celecoxib, and COX 2 inhibitors which reduce inflammation, analgesics (acetaminophen, oxycontin) which reduce pain, and a limited number of actual biological modifiers are in use (e.g. etanercept, infliximab commercially known as Enbrel and Remicade, respectively). Thus, new and more effective treatments are currently needed to relieve the symptoms and slow the progression of rheumatoid arthritis and other chronic arthridites.

In addition to inflammation, the joints of chronic rheumatoid arthritis patients have been shown to have marked growth of synovial cells, formation of a multilayer structure due to abnormal growth of the synovial cells (pannus formation), invasion of the synovial cells into cartilage tissue and bone tissue, vascularization toward the synovial tissue, and infiltration of inflammatory cells such as lymphocytes and macrophages which is supported by the lymphatic vasculature.

The current state of the art demonstrates that treatments for patients with RA are needed which are not general immune suppressants, do not result in serious side effects and which target different aspects of rheumatoid arthritis.

### SUMMARY OF THE INVENTION

The present invention relates to a method for screening individuals exhibiting symptoms of chronic arthridites for increased levels of VEGF-C ligand and the expression of its receptor, VEGFR-3, in synovial sites. The present invention further contemplates administering therapeutic compounds to subjects exhibiting symptoms of arthritis and elevated levels of VEGF-C protein expression, wherein said therapeutic compounds prevent the interactions of VEGF-C with its receptor VEGFR-3.

In one embodiment the invention provides a method of treating a mammalian subject with chronic arthridites, comprising the steps of screening a mammalian subject with symptoms of chronic arthridites for VEGF-C protein expression in synovial sites, and administering to a mammalian subject identified by the screening as having elevated VEGF-C expression in the synovium a composition comprising an inhibitor of VEGFR-3 in an amount effective to ameliorate symptoms of chronic arthridites. In one embodiment, the chronic arthridites is rheumatoid arthritis. In a preferred embodiment, the mammalian subject is human.

In the method of the invention, patients with any chronic arthridites are candidates for screening and therapy for elevated VEGF-C expression, including patients having rheumatoid arthritis, osteoarthritis, juvenile arthritis, ankylosing spondylosis, HIV-related arthritis and psoriatic arthritis.

Practice of methods of the invention in other mammalian subjects, especially mammals that are conventionally used as models for demonstrating therapeutic efficacy in humans (e.g., primate, porcine, canine, or rabbit animals), is also contemplated.

In one embodiment, the screening step of the invention comprises obtaining a biological sample from a synovial site of the mammalian subject with symptoms of chronic arthridities and measuring VEGF-C polypeptide expression in the biological sample to identify elevated VEGF-C expression. VEGF-C levels can be measured from isolated synovial fluid samples by standard in vitro techniques well-known in the art, such as enzyme-linked immunosorbant assay (ELISA), radio immunoassay (RIA), Northern hybridization or quantitative RT-PCR. VEGF-C can also be measured in synovial tissue samples using fluorescent microscopy with fluorescently labeled anti-VEGF-C antibodies. The determination of “elevated VEGF-C” is made relative to VEGF-C expression levels in the same type of tissue of patients not exhibiting symptoms of chronic arthridites. As with all medical diagnoses, it will be appreciated that levels of VEGF-C expression may vary within healthy or diseased populations based on sex, age, ethnicity and other factors. Still, chronic arthridites patients that exhibit higher VEGF-C mRNA or protein expression than is observed in healthy tissue are readily identified as candidates for VEGFR-3 inhibitor therapy. The detection is correlated, for example, by a brighter staining signal in a fluorescent microscopy assay, the presence of more staining in a fluorescent microscopy assay, or by elevated fluid levels of VEGF-C as detected by PCR, ELISA or RIA. If desired, measurement from a population of patients can be analyzed using standard statistics to identify cut-off measurements of VEGF-C that represent statistically significant elevation relative to appropriately matched healthy controls.

In one embodiment the biological sample comprises synovial tissue, e.g., from joints affected by symptoms of arthritis, or synovial fluid, i.e. the fluid that results in joint swelling.

In another embodiment, the screening step comprises administering to a mammalian subject with symptoms of chronic arthridites a composition comprising an antibody or antibody fragment that specifically binds VEGF-C and determining VEGF-C protein expression based on the quantity or distribution of said antibody in the mammalian subject, wherein an increased level of VEGF-C expression in synovial sites correlates with the presence of chronic arthridites. The method alternatively comprises (instead of the administering step) the step of obtaining a biological sample of synovial fluid or synovial tissue from said mammalian subject, contacting the sample with the antibody, and determining the quantity and/or distribution of VEGF-C in the biological sample, wherein an elevated level of VEGF-C expression correlates with the presence of chronic arthridites.

For this method, the antibody or antibody fragment can further comprise a label. The label attached to the antibody or antibody fragment can be a radiolabel such as ¹⁴C, ¹³³I, ¹²⁵I, Barium isotopes, or Indium III, or a colorimetric label such as fluorescein, phycobiliprotien; tetraethyl rhodamine; or enzymes which produce a fluorescent or colored product for detection by fluorescence; absorbance, or visible color.

The VEGFR-3 inhibitor can be any molecule that acts with specificity to reduce VEGF-C/VEGFR-3 interaction, e.g., by blocking VEGF-C binding to VEGFR-3 or by reducing expression of VEGF-C or VEGFR-3. In a preferred embodiment, the VEGFR-3 inhibitor inhibits VEGF-C binding to VEGFR-3. The VEGFR-3 inhibitor administered to a subject identified in the screening step can be a polypeptide comprising a soluble VEGFR-3 polypeptide fragment that binds to VEGF-C protein, VEGFR-3 anti-sense polynucleotides or short-interfering RNA (siRNA), an anti-VEGFR-3 antibody, a polypeptide comprising an antigen binding fragment of an anti-VEGFR-3 antibody and an anti-VEGF-C antibody.

In one embodiment, the VEGFR-3 inhibitor comprises a soluble VEGFR-3 polypeptide fragment comprising an extracellular domain fragment of mammalian VEGFR-3, wherein said fragment binds to VEGF-C protein. Preferably the VEGFR-3 fragment is human. In one variation, the extracellular domain fragment comprises immunoglobulin domains one through three of VEGFR-3. In a preferred embodiment, the extracellular domain fragment contemplated by the invention comprises amino acids 33 to 324 of human VEGFR-3 set out in SEQ ID NO: 4. In an alternate embodiment, the soluble VEGFR-3 fragment is linked to an immunoglobulin Fc domain.

In one embodiment, the inhibitor composition contemplated by the method comprises a polypeptide comprising an amino acid sequence comprising at least 90%, 95%, 96%, 97%, 98%, or 99% amino acid identity to amino acids 33-324 of human VEGFR-3 set out in SEQ ID NO: 4 and maintains ligand binding activity of human VEGFR-3.

In an additional embodiment, the VEGFR-3 inhibitor composition comprises a polypeptide encoded by a polynucleotide that hybridizes to the complement of amino acids 33 to 324 of SEQ. ID NO 4 under either moderate or highly stringent conditions. Exemplary moderately stringent conditions of hybridization are hybridization in 0.5 M NaHPO4, 7% sodium dodecyl sulfate (SDS), 1 mM EDTA at 65° C. and washing in 0.2×SSC/0.1% SDS at 42°. Exemplary highly stringent hybridization conditions are: 0.5 M NaHPO₄, 7% sodium dodecyl sulfate (SDS), 1 mM EDTA at 65° C. and washing in 0.1×SSC/0.1% SDS at 68° C. It is understood in the art that conditions of equivalent stringency can be achieved through variation of temperature and buffer, or salt concentration as described Ausubel et al. (Eds.), Current Protocols in Molecular Biology, John Wiley & Sons (1994), pp. 6.0.3-6.4.10.

VEGFR-3 antisense nucleic acid molecules for use in the method comprise a sequence complementary to any integer number of nucleotides from the target sequence from about 10 to 500, preferably an integer number from 10 to 50. In exemplary embodiments, a VEGFR-3 antisense molecule comprises a complementary sequence at least about 10, 25, 50, 100, 250 or 500 nucleotides in length or complementary to an entire VEGFR-3 coding strand. More specifically, antisense molecules of 10, 15, 20, 25, 30, 35, 40, 45, or 50 nucleotides in length are contemplated.

The siRNAs contemplated for use in the invention provide both a sense and antisense coding strand of the VEGFR-3 mRNA. siRNAs are typically 30 nucleotides or less in length, and more preferably 21- to 23-nucleotides, with characteristic 2- to 3-nucleotide 3′-overhanging ends, which are generated by ribonuclease III cleavage from longer dsRNAs.

Additional VEGFR-3 inhibitors contemplated for use in the method include anti-VEGFR-3 and anti-VEGF-C antibodies. Anti-VEGFR-3 antibodies for use in the method comprise either fully intact anti-VEGFR-3 antibodies, or an antigen binding fragment of the anti-VEGFR-3 antibody. Antigen binding regions of the anti-VEGFR-3 antibody include Fab, Fab′, F(ab′)₂, and Fv polypeptides. Anti-VEGF-C antibodies that bind to VEGF-C and thereby inhibit the binding of the VEGF-C ligand to VEGFR-3 are also contemplated. Such compounds also include polypeptides that comprise antigen binding fragments of anti VEGF-C antibodies.

In preferred embodiments, the composition to be administered further comprises a pharmaceutically acceptable diluent, adjuvant or carrier medium such as water, saline, phosphate-buffered saline, glucose, or other carriers conventionally used to deliver therapeutics to an individual identified by a screening method as a treatment candidate.

In another variation, the invention contemplates a method of treating a mammal having chronic arthridites characterized by elevated levels of VEGF-C protein expression at synovial sites, comprising a step of administering to said mammalian organism a composition, said composition comprising a VEGFR-3 inhibitor which inhibits binding between VEGF-C and VEGFR-3 expressed in cells of said organism, thereby inhibiting VEGFR-3 function. In one embodiment, the chronic arthridites is rheumatoid arthritis. In a preferred embodiment, the mammalian subject is human.

The method optionally further comprises a screening step preceding the administering step, wherein the screening step comprises screening a human with symptoms of chronic arthridites to identify a chronic arthridites characterized by increased VEGF-C protein expression. When a screening step is included, the administering step comprises administering the VEGFR-3 inhibitory composition to a human identified by the screening step as having chronic arthridites characterized by elevated levels of VEGF-C protein expression.

In a preferred embodiment, the VEGFR-3 inhibitor inhibits VEGF-C binding to VEGFR-3. As described in detail above, the VEGFR-3 inhibitor administered to a subject identified in the screening step can be a polypeptide comprising a soluble VEGFR-3 polypeptide fragment that binds to VEGF-C protein, VEGFR-3 anti-sense polynucleotides or siRNA, an anti-VEGFR-3 antibody, a polypeptide comprising an antigen-binding fragment of an anti-VEGFR-3 antibody, an anti-VEGF-C antibody, and/or a polypeptide comprising an antigen-binding fragment of an anti-VEGF-C antibody.

Further contemplated by the invention is a method wherein the VEGFR-3 inhibitory composition is administered in combination with a medication intended to alleviate symptoms of chronic arthritis. The VEGFR-3 composition can be administered in combination with therapeutics such as non-steroidal anti-inflammatory drugs (NSAIDs), analgesiscs, glucocoritcoids, disease-modifying antirheumatic drugs (DMARDs) or biologic response modifiers.

Exemplary NSAIDs are chosen from the group consisting of ibuprofen, naproxen, naproxen sodium, Cox-2 inhibtors such as Vioxx and Celebrex, and sialylates. Exemplary analgesics are chosen from the group consisting of acetaminophen, oxycodone, tramadol of proporxyphene hygrochloride. Exemplary glucocorticouids are chosen from the group consisting of cortisone, dexamethosone, hydrocortisone, methylprednisolone, prednisolone, or prednisone. Exemplary biological response modifiers are selected from the group consisting of etanercept (Enbrel) or infliximab (remicade). Exemplary DMARDs are selected from the group consisting of auranofin, azathioprine, cyclophosphamide, cyclosporine, methotrexate, or penicillamine. Formulations comprising one or more inhibitors of the invention and one or more of the foregoing conventional therapeutics also are contemplated as an aspect of the invention.

The administering of the methods can be performed either systemically or locally at synovial sites. Exemplary systemic administrations may include intravenous administration, oral routes, and sustained delivery or sustained release mechanisms, which can deliver the formulation internally. For example, biodegradeable microspheres or capsules or other biodegradeable polymer configurations capable of sustained delivery of a composition (e.g., a chimeric molecule) can be included in the formulations of the invention (see, e.g., Putney Nat. Biotechnol. (1998) 16: 153-157). Compositions administered locally at the site of synovial VEGF-C expression can be via injection (e.g. sub-cutaneous or intra-articular), topical application and other methods of local administration.

Additional features and variations of the invention will be apparent to those skilled in the art from the entirety of this application, including the detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

In addition to the foregoing, the invention includes, as an additional aspect, all embodiments of the invention narrower in scope in any way than the variations specifically mentioned above. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### DETAILED DESCRIPTION OF THE INVENTION

The present invention relates to the treatment of chronic arthridites by modulating the interaction of VEGF-C polypeptides with the tyrosine kinase receptor VEGFR-3, both of which have been detected in the synovium of patients with rheumatoid arthritis, an exemplary chronic arthritic disease.

VEGF-C and VEGFR-3 are members of a complex network of growth factors and receptors involved in several areas of development known as the PDGF/VEGF and PDGFR/VEGFR family proteins. The PDGF subfamily is reviewed in Heldin et al., Biochimica et Biophysica Acta 1378:F79-113 (1998).

The VEGF subfamily, which includes VEGF or VEGF-A, VEGF-B, VEGF-C, VEGF-D and various structural isoforms of each protein, is composed of PDGF/VEGF members which share a VEGF homology domain (VHD) characterized by the sequence: C-X(22-24)-P-[PSR]-C-V-X(3)-R-C-[GSTA]-G-C-C-X(6)-C-X(32-41)-C.

The growth factor Vascular Endothelial Growth Factor C (VEGF-C), as well as native human, non-human mammalian, and avian polynucleotide sequences encoding VEGF-C, and VEGF-C variants and analogs, have been described in detail in International Patent Application Number PCT/US98/01973, filed Feb. 2, 1998 and published on Aug. 6, 1998 as International Publication Number WO 98/33917; in Joukov et al., J. Biol. Chem., 273(12): 6599-6602 (1998); and in Joukov et al., EMBO J., 16(13): 3898-3911 (1997), all of which are incorporated herein by reference in their entirety. As explained therein in detail, human VEGF-C is initially produced in human cells as a prepro-VEGF-C polypeptide of 419 amino acids. A cDNA and deduced amino acid sequence for human prepro-VEGF-C are set forth in SEQ ID NOs: 1 and 2, respectively, and a cDNA encoding human VEGF-C has been deposited with the American Type Culture Collection (ATCC), 10801 University Blvd.; Manassas, Va. 20110-2209 (USA), pursuant to the provisions of the Budapest Treaty (Deposit date of 24 Jul. 1995 and ATCC Accession Number 97231). VEGF-C sequences from other species also have been reported. See Genbank Accession Nos. MMU73620 (Mus musculus); and CCY15837 (Coturnix coturnix) for example, incorporated herein by reference.

The prepro-VEGF-C polypeptide is processed in multiple stages to produce a mature and most active VEGF-C polypeptide of about 21-23 kD (as assessed by SDS-PAGE under reducing conditions). Such processing includes cleavage of a signal peptide (SEQ ID NO: 2, residues 1-31); cleavage of a carboxyl-terminal peptide (corresponding approximately to amino acids 228-419 of SEQ ID NO: 2 and having a pattern of spaced cysteine residues reminiscent of a Balbiani ring 3 protein (BR3P) sequence [Dignam et al., Gene, 88:133-40 (1990); Paulsson et al., J. Mol. Biol., 211:331-49 (1990)]) to produce a partially-processed form of about 29 kD; and cleavage (apparently extracellularly) of an amino-terminal peptide (corresponding approximately to amino acids 32-103 of SEQ ID NO: 2) to produced a fully-processed mature form of about 21-23 kD. Experimental evidence demonstrates that partially-processed forms of VEGF-C (e.g., the 29 kD form) are able to bind VEGFR-3 (Flt4 receptor), whereas high affinity binding to VEGFR-2 occurs only with the fully processed forms of VEGF-C. It appears that VEGF-C polypeptides naturally associate as non-disulfide linked dimers.

VEGF-C is involved in the regulation of lymphangiogenesis: when VEGF-C was overexpressed in the skin of transgenic mice, a hyperplastic lymphatic vessel network was observed, suggesting that VEGF-C induces lymphatic growth [Jeltsch et al., Science, 276:1423-1425 (1997)].

The PDGF receptors are protein tyrosine kinase receptors (PTKs) that contain five immunoglobulin-like loops in their extracellular domains. VEGFR-1, VEGFR-2, and VEGFR-3 comprise a subgroup of PTKs distinguished by the presence of seven Ig domains in their extracellular domain and a split kinase domain in the cytoplasmic region.

Structural analyses of the VEGF receptors indicate that the VEGF-A binding site on VEGFR-1 and VEGFR-2 is located in the second and third Ig-like loops. Similarly, the VEGF-C and VEGF-D binding sites on VEGFR-2 and VEGFR-3 are also contained within the first to third Ig-loops [Taipale et al., Curr Top Microbiol Immunol 237:85-96 (1999)]. It has been demonstrated that the second Ig-like loop confers ligand specificity as shown by domain swapping experiments [Ferrara, J Mol Med 77:527-543 (1999)]. Receptor-ligand studies indicate that dimers formed by the VEGF family proteins are capable of binding two VEGF receptor molecules, thereby dimerizing VEGF receptors.

VEGFR-3 is expressed broadly in endothelial cells during early embryogenesis. See e.g. U.S. Pat. No. 5,776,755, U.S. Pat. No. 6,107,046, WO 02/057299, and WO02/060950. During later stages of development, the expression of VEGFR-3 becomes restricted to developing lymphatic vessels [Kaipainen, et al., Proc. Natl. Acad. Sci. USA, 92: 3566-3570 (1995)]. In adults, the lymphatic endothelia, certain fenestrated endothelia (Partanen, T. et al. FASEB J. 14: 2087-2096, 2000) and some high endothelial venules express VEGFR-3, and increased expression occurs in lymphatic sinuses in metastatic lymph nodes and in lymphangioma. VEGFR-3 is also expressed in a subset of CD34+hematopoietic cells which may mediate the myelopoietic activity of VEGF-C demonstrated by overexpression studies [WO 98/33917]. Targeted disruption of the VEGFR-3 gene in mouse embryos leads to failure of the remodeling of the primary vascular network, and death after embryonic day 9.5 [Dumont et al., Science, 282: 946-949 (1998)]. These studies suggest an essential role for VEGFR-3 in the development of the embryonic vasculature, and also during lymphangiogenesis. In adult tissues VEGFR-3 expression occurs mainly in the lymphatic endothelia (Kaipainen et al., Proc. Natl. Acad. Sci. USA, 92: 3566-3570, 1995; Partanen et al., FASEB J., 14:2087-2096, 2000), and VEGFR-3 ligands VEGF-C and VEGF-D can induce growth of the lymphatic vessels (Jeltsch et al., Science, 276:1423-1425, 1997; Veikkola et al., EMBO J. 20: 1223-1231, 2001). In contrast, blocking of VEGFR-3 signaling by use of a soluble VEGFR-3 protein caused regression of developing lymphatic vessels by inducing endothelial cell apoptosis (Makinen et al., Nature Med. 7:199-205, 2001).

VEGFR-3 Derivatives, Analogues and Peptides

In one embodiment, a therapeutic or prophylactic treatment of chronic arthridites provided by the present invention involves administering to a mammalian subject, such as a human, a composition comprising a VEGFR-3 inhibitory compound such as a suitable VEGFR-3 polynucleotide or polypeptide or combination thereof (sometimes generically referred to herein as a “VEGFR-3 composition” or “VEGFR-3 inhibitor(y) composition” or “VEGFR-3 inhibitor”).

By “VEGFR-3 inhibitory compound” is meant any compound that specifically inhibits the growth factor mediated signaling of the VEGFR-3 polypeptide by blocking ligand-receptor binding, receptor activation or blocking ligand or receptor expression. It is contemplated that such compounds that inhibit ligand-receptor binding will be effective to inhibit the binding of VEGF-C to VEGFR-3. Exemplary VEGFR-3 inhibitor compounds include the following: (a) a polypeptide comprising a soluble VEGFR-3 fragment (e.g., an extracellular domain fragment), wherein the fragment and the polypeptide are capable of binding to a VEGFR-3 ligand; (b) an anti-VEGFR-3 antibody; (c) a polypeptide comprising an antigen binding fragment of an anti VEGFR-3 antibody; (d) a polypeptide comprising a fragment or analog of a vertebrate vascular endothelial growth factor C (VEGF-C) polypeptide, wherein the polypeptide and the fragment or analog bind, but fail to activate, the VEGFR-3 expressed on native host cells (i.e., cells of the organism that express the native VEGFR-3 protein on their surface); (e) an anti-VEGF-C antibody, (f) a polypeptide comprising an antigen-binding fragment of an anti-VEGF-C antibody (g) a VEGFR-3 antisense polynucleotide or siRNA, (h) a VEGF-C antisense polynucleotide or siRNA and (i) a VEGFR-3 tyrosine kinase inhibitor. Small molecule inhibitors identifiable by standard in vitro screening assays, e.g., using VEGF-C and recombinantly expressed VEGFR-3 also are contemplated. Polypeptides comprising an antigen binding fragment of an anti-VEGFR-3 antibody are highly preferred. Such polypeptides include, e.g., polyclonal and monoclonal antibodies that specifically bind VEGFR-3; fragments of such antibodies; chimeric and humanized antibodies, human antibodies and the like. Use of compounds that bind to circulating VEGFR-3 ligand and thereby inhibit the binding of the ligand to VEGFR-3 also is contemplated. Such compounds include anti-VEGF-C antibodies or polypeptides that comprise antigen binding fragments thereof. In a related variation, the invention contemplates methods of treatment that disrupt downstream intracellular VEGFR-3 signaling, thereby inhibiting VEGFR-3 function.

“Inhibitory effect” when used in reference to the activity of a VEGFR-3 inhibitory compound contemplated by the present invention means that the VEGFR-3 inhibitor substantially inhibits the activity of VEGF-C. Generally, the result of this inhibitory effect is a decrease in pathogenic lymphangiogenesis or angiogenesis which occurs in chronic arthridites as a result of the VEGF-C protein.

For treatment of humans, VEGFR-3 polypeptides with an amino acid sequence of a human VEGFR-3 are highly preferred, and polynucleotides comprising a nucleotide sequence of a human VEGFR-3 cDNA are highly preferred. By “human VEGFR-3” is meant a polypeptide corresponding to a naturally occurring protein (encoded by any allele of the human VEGFR-3 gene), or a polypeptide comprising a biologically active fragment of a naturally-occurring mature protein. By way of example, a human VEGFR-3 comprises a continuous portion of the amino acid sequence set forth in SEQ ID NO: 4 sufficient to permit the polypeptide to bind VEGF-C, wherein the human VEGFR-3 is a soluble, extracellular fragment of the VEGFR-3 polypeptide. For instance, the VEGF-C binding site on the VEGFR-3 polypeptide is contained within the second immunoglobulin domain region of the polypeptide [Taipale et al., supra]. An exemplary human VEGFR-3 polypeptide fragment used by the invention incorporates the immunoglobulin domain containing the ligand binding site and flanking immunogloubulin regions to facilitate binding of the human VEGFR-3 fragment to its ligand. Thus, in a preferred embodiment, the human VEGFR-3 fragment is a soluble fragment which comprises a portion of the extracellular domain of the VEGFR-3 polypeptide. In an alternate embodiment, the soluble VEGFR-3 fragment is fused with an immunoglobulin Fc domain or other fusion partner, wherein the fusion protein exhibits a longer half-life in the serum of the mammalian subject.

Also contemplated as VEGFR-3 polypeptides are non-human mammalian or avian VEGFR-3 polypeptides and polynucleotides. By “mammalian VEGFR-3” is meant a polypeptide corresponding to a naturally occurring protein encoded by any allele of a VEGFR-3 gene of any mammal, or a polypeptide comprising a biologically active fragment of a mature protein. The term “mammalian VEGFR-3 polypeptide” is intended to include analogs of mammalian VEGFR-3's that possess the in vivo VEGFR-3 biological activity of the mammalian VEGFR-3. Examplary mammalian and avian VEGFR-3 mRNA sequences include Genbank Accession No. NM_{—}008029. (mouse), Genbank Accession No. NM_{—}053652. (rat), Genbank Accession No. AF453570. (rabbit) and Genbank Accession No. AF041795. (chicken).

Because the recombinant techniques can be used to make therapeutic VEGFR-3 fragment, it is within the skill in the art to make and use analogs of human VEGFR-3 (and polynucleotides that encode such analogs) wherein one or more amino acids have been added, deleted, or replaced with other amino acids, especially with conservative replacements, and wherein the VEGF-C binding biological activity has been retained. Analogs that retain VEGF-C binding are contemplated as VEGFR-3 polypeptides for use in the present invention. In a preferred embodiment, analogs having 1, 2, 3, 4, 5, 6, 7, 8, 9, 10, 11, 12, 13, 14, 15, 16, 17, 18, 19, 20, 21, 22, 23, 24, or 25 such modifications and that retain VEGFR-3 ligand binding activity are contemplated as VEGFR-3 inhibitory polypeptides for use in the present invention. Polynucleotides encoding such analogs are generated using conventional PCR, site-directed mutagenesis, and chemical synthesis techniques.

For many proteins, the effects of any individual or small group of amino acid changes is unlikely to significantly alter biological properties, especially if the changes are conservative substitutions, provided the changes are not introduced at critical residues. Preferred variants of polypeptides used in the invention (e.g., VEGFR-3 fragments) share at least about 70%, 80%, 90%, 95%, 96%, 97%, 98%, or 99% amino acid identity with hybrids that consist entirely of amino acid sequences derived from naturally occurring VEGFR-3.

It is well known in the literature to recombinantly express proteins with an initiator methionine, with a heterologous signal peptide, with one or more tag sequences to facilitate purification, as fusions with other polypeptides, and the like. It is also well known to modify polypeptides with glycosylation, pegylation, or other modifications, some of which improve stability, circulating half-life, or (in the case of glycosylation) may make the polypeptide more similar to endogenous vascular endothelial growth factors. Polypeptides fragments for use according to the invention may comprise any such modifications and additions to the amino acid sequence derived from a naturally-occurring vertebrate VEGFR-3 polypeptide fragment.

A “functional derivative” of a VEGFR-3 inhibitor is a polypeptide which possesses an activity that is substantially similar to a biological activity of non-recombinant VEGFR-3 inhibitor compound. A functional derivative of the VEGFR-3 inhibitor may or may not contain post-translational modifications such as covalently linked carbohydrate, depending on the necessity of such modifications for the performance of a the VEGFR-3 inhibitory function. The term “functional derivative” is intended to include the “fragments,” “variants,” “analogues,” and “chemical derivatives” of a molecule.

As used herein, a molecule is said to be a “chemical derivative” of another molecule when it contains additional chemical moieties not normally a part of the molecule. Such moieties may improve the molecule's solubility, absorption, biological half-life, etc. The moieties may alternatively decrease the toxicity of the molecule and eliminate or attenuate any undesirable side effect of the molecule, etc. Moieties capable of mediating such effects are disclosed in Remington's Pharmaceutical Sciences (1980). Procedure for coupling such moieties to a molecule are well known in the art.

A “fragment” of a molecule such as VEGFR-3 polypeptide is meant to refer to any portion of the molecule, such as the peptide core, a variant of the peptide core, or an extracellular region of the polypeptide.

A “variant” of a molecule such as VEGFR-3 polypeptide is meant to refer to a molecule substantially similar in structure and biological activity to either the entire molecule, or to a fragment thereof. Thus, provided that two molecules possess a similar activity, they are considered variants as that term is used herein even if the composition or secondary, tertiary, or quaternary structure of one of the molecules is not identical to that found in the other, or if the sequence of amino acid residues is not identical.

An “analogue” of VEGFR-3 polypeptide or genetic sequence is meant to refer to a protein or genetic sequence substantially similar in function and structure to the VEGFR-3 polypeptide or genetic sequence set out herein in SEQ ID NOs: 3 and 4.

An alignment of human VEGFR-3 with VEGFR-3 from other species (performed using any generally accepted alignment algorithm) suggests additional residues wherein modifications can be introduced (e.g., insertions, substitutions, and/or deletions) without destroying VEGFR-3 ligand binding activity. Any position at which aligned VEGFR-3 polypeptides of two or more species have different amino acids, especially different amino acids with side chains of different chemical character, is a likely position susceptible to modification without concomitant elimination of function.

Apart from the foregoing considerations, it will be understood that conservative amino acid substitutions can be performed to a wildtype VEGFR-3 sequence which are likely to result in a polypeptide that retains VEGFR-3 biological activities, especially if the number of such substitutions is small. By “conservative amino acid substitution” is meant substitution of an amino acid with an amino acid having a side chain of a similar chemical character. Similar amino acids for making conservative substitutions include those having an acidic side chain (glutamic acid, aspartic acid); a basic side chain (arginine, lysine, histidine); a polar amide side chain (glutamine, asparagine); a hydrophobic, aliphatic side chain (leucine, isoleucine, valine, alanine, glycine); an aromatic side chain (phenylalanine, tryptophan, tyrosine); a small side chain (glycine, alanine, serine, threonine, methionine); or an aliphatic hydroxyl side chain (serine, threonine). Addition or deletion of one or a few internal amino acids without destroying VEGFR-3 biological activities also is contemplated.

Derivatives, analogues, or peptides may have enhanced ligand binding activity in comparison to native VEGFR-3 polypeptide fragments, depending on the particular application. VEGFR-3 related derivatives, analogues, and peptides of the invention may be produced by a variety of means known in the art. Procedures and manipulations at the genetic and protein levels are within the scope of the invention. Peptide synthesis, which is standard in the art, may be used to obtain VEGFR-3 peptides. At the protein level, numerous chemical modifications may be used to produce VEGFR-3-like derivatives, analogues, or peptides by techniques known in the art, including but not limited to specific chemical cleavage by endopeptidases (e.g. cyanogen bromides, trypsin, chymotrypsin, V8 protease, and the like) or exopeptidases, acetylation, formylation, oxidation, etc.

Preferred derivatives, analogs, and peptides are those which retain VEGFR-3 ligand binding activity. Those derivatives, analogs, and peptides which bind VEGFR-3 ligand but do not transduce a signal in response thereto are useful as VEGFR-3 inhibitors. A preferred VEGFR-3 ligand for use in such binding and/or autophosphorylation assays when screening for inhibitors is a ligand comprising an approximately 23 kd polypeptide that is isolatable from a PC-3 conditioned medium as described herein. This ligand, designated VEGF-C, has been characterized in detail in PCT Patent Application PCT/FI96/00427, filed Aug. 1, 1996, and published as International Publication WO 97/05250, and in U.S. patent application Ser. No. 08/671,573 all of which are incorporated herein by reference in their entirety.

A VEGFR-3-Ig fusion construct, a recombinant DNA encoding an VEGFR-3-immunoglobulin chimera, is constructed as described in U.S. patent application Ser. No. 09/765,534 (incorporated herein by reference). Briefly, a VEGFR-3 (Flt4) extracellular (EC) domain fragment consisting of the first three Ig domains of VEGFR-3 (encoded by nucleotides 20-1005 of GenBank Acc. No. X68203, SEQ. ID NO.: 3) is ligated into the LTR-FLT41 vector replacing the sequences encoding the transmembrane and cytoplasmic domains. This Flt4EC insert containing a splice donor site was ligated first into pH*CE2 containing exons encoding the human immunoglobulin heavy chain hinge and constant region exons (Karjalainen, K., TIBTECH, 9: 109-113 (1991)). The EcoRI-Bam HI insert containing the Flt4-Ig chimera was then blunted by methods standard in the art (Klenow) and ligated to the blunted HindIII site in pREP7 (Invitrogen). The construct was transfected into 293-EBNA T cells by the calcium-phosphate precipitation method and the conditioned medium was used for the isolation of the Flt4-Ig protein by protein A-Sepharose affinity chromatography.

Anti-VEGFR-3 (Flt4) Antibodies

Previously, a number of VEGFR-3 antibodies have been described, see for example, U.S. Pat. No. 6,107,046 (incorporated herein by reference).

Antibodies are useful for modulating VEGFR-3/VEGF-C interactions due to the ability to easily generate antibodies with relative specificity, and due to the continued improvements in technologies for adopting antibodies to human therapy. Thus, the invention contemplates use of antibodies (e.g., monoclonal and polyclonal antibodies, single chain antibodies, chimeric antibodies, bifunctional/bispecific antibodies, humanized antibodies, human antibodies, and complementary determining region (CDR)-grafted antibodies, including compounds which include CDR sequences which specifically recognize a polypeptide of the invention) specific for polypeptides of interest to the invention, especially VEGFR-3 and VEGF-C proteins. Preferred antibodies are human antibodies which are produced and identified according to methods described in WO93/11236, published Jun. 20, 1993, which is incorporated herein by reference in its entirety. Antibody fragments, including Fab, Fab′, F(ab′)2, and Fv, are also provided by the invention. The term “specific for,” when used to describe antibodies of the invention, indicates that the variable regions of the antibodies of the invention recognize and bind the polypeptide of interest exclusively (i.e., able to distinguish the polypeptides of interest from other known polypeptides of the same family, by virtue of measurable differences in binding affinity, despite the possible existence of localized sequence identity, homology, or similarity between family members). It will be understood that specific antibodies may also interact with other proteins (for example, S. aureus protein A or other antibodies in ELISA techniques) through interactions with sequences outside the variable region of the antibodies, and in particular, in the constant region of the molecule. Screening assays to determine binding specificity of an antibody of the invention are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies of the invention can be produced using any method well known and routinely practiced in the art.

Various procedures known in the art may be used for the production of polyclonal antibodies to epitopes of VEGFR-3 (See U.S. Pat. No. 6,107,046). For the production of antibodies, various host animals (including but not limited to rabbits, mice, rats, hamsters, etc.) can be immunized by injection with VEGFR-3, or a synthetic VEGFR-3 peptide. Various adjuvants may be used to increase the immunological response, depending on the host species, including but not limited to Freund's (complete and incomplete) adjuvant, mineral gels such as aluminium hydroxide, surface active substances such as lysolecithin, pluronic polyols, polyanions, oil emulsions, keyhole limpet hemocyanins, dinitrophenol, and potentially useful human adjuvants such as BCG (Bacillus Calmette-Guerin) and Corynebacterium parvum.

Briefly, a polyclonal antibody is prepared by immunizing an animal with an immunogen comprising a polypeptide of the present invention and collecting antisera from that immunized animal. A wide range of animal species can be used for the production of antisera. Typically an animal used for production of anti-antisera is a non-human animal including rabbits, mice, rats, hamsters, goat, sheep, pigs or horses. Because of the relatively large blood volume of rabbits, a rabbit is a preferred choice for production of polyclonal antibodies.

A monoclonal antibody to an epitope of VEGFR-3 may be prepared by using any technique which provides for the production of antibody molecules by continuous cell lines in culture. These include but are not limited to the hybridoma technique originally described by Köhler et al., Nature, 256: 495-497 (1975), and the more recent human B-cell hybridoma technique [Kosbor et al., Immunology Today, 4: 72 (1983)] and the EBV-hybridoma technique [Cole et al., Monoclonal Antibodies and Cancer Therapy, Alan R Liss, Inc., pp. 77-96 (1985)]. Antibodies against VEGFR-3 also may be produced in bacteria from cloned immunoglobulin cDNAs. With the use of the recombinant phage antibody system it may be possible to quickly produce and select antibodies in bacterial cultures and to genetically manipulate their structure.

When the hybridoma technique is employed, myeloma cell lines may be used. Such cell lines suited for use in hybridoma-producing fusion procedures preferably are non-antibody-producing, have high fusion efficiency, and enzyme deficiencies that render them incapable of growing in certain selective media which support the growth of only the desired fused cells (hybridomas). For example, where the immunized animal is a mouse, one may use P3-X63/Ag8, P3-X63-Ag8.653, NS1/1.Ag 41, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions. It should be noted that the hybridomas and cell lines produced by such techniques for producing the monoclonal antibodies are contemplated to be novel compositions of the present invention.

In an exemplary method for generating a polyclonal antisera immunoreactive with the chosen VEGFR-3 epitope, 50 μg of VEGFR-3 antigen is emulsified in Freund's Complete Adjuvant (CFA) for immunization of rabbits. At intervals of, for example, 21 days, 50 μg of epitope are emulsified in Freund's Incomplete Adjuvant for boosts

To generate monoclonal antibodies, a mouse is injected periodically with recombinant VEGFR-3 against which the antibody is to be raised (e.g., 10-20 μg emulsified in Freund's Complete Adjuvant). The mouse is given a final pre-fusion boost of a VEGFR-3 polypeptide containing the epitope that allows specific recognition of lymphatic endothelial cell in phosphate buffered saline (PBS), and four days later the mouse is sacrificed and its spleen removed. The spleen is placed in 10 ml serum-free RPMI 1640, and a single cell suspension is formed by grinding the spleen between the frosted ends of two glass microscope slides submerged in serum-free RPMI 1640, supplemented with 2 mM L-glutamine, 1 mM sodium pyruvate, 100 units/ml penicillin, and 100 μg/ml streptomycin (RPMI) (Gibco, Canada). The cell suspension is filtered through sterile 70-mesh Nitex cell strainer (Becton Dickinson, Parsippany, N.J.), and is washed twice by centrifuging at 200 g for 5 minutes and resuspending the pellet in 20 ml serum-free RPMI. Splenocytes taken from three naive Balb/c mice are prepared in a similar manner and used as a control. NS-1 myeloma cells, kept in log phase in RPMI with 11% fetal bovine serum (FBS) (Hyclone Laboratories, Inc., Logan, Utah) for three days prior to fusion, are centrifuged at 200 g for 5 minutes, and the pellet is washed twice as described in the foregoing paragraph.

1×10⁸ spleen cells are combined with 2.0×10⁷ NS-1 cells and centrifuged, and the supernatant is aspirated. The cell pellet is dislodged by tapping the tube, and 1 ml of 37° C. PEG 1500 (50% in 75 mM Hepes, pH 8.0) (Boehringer Mannheim) is added with stirring over the course of 1 minute, followed by the addition of 7 ml of serum-free RPMI over 7 minutes. An additional 8 ml RPMI is added and the cells are centrifuged at 200 g for 10 minutes. After discarding the supernatant, the pellet is resuspended in 200 ml RPMI containing 15% FBS, 100 μM sodium hypoxanthine, 0.4 μM aminopterin, 16 μM thymidine (HAT) (Gibco), 25 units/ml IL-6 (Boehringer Mannheim) and 1.5×10⁶ splenocytes/ml and plated into 10 Corning flat-bottom 96-well tissue culture plates (Corning, Corning N.Y.).

On days 2, 4, and 6, after the fusion, 100 μl of medium is removed from the wells of the fusion plates and replaced with fresh medium. On day 8, the fusion is screened by ELISA, testing for the presence of mouse IgG binding to VEGFR-3 as follows. Immulon 4 plates (Dynatech, Cambridge, Mass.) are coated for 2 hours at 37° C. with 100 ng/well of VEGFR-3 diluted in 25 mM Tris, pH 7.5. The coating solution is aspirated and 200 ul/well of blocking solution (0.5% fish skin gelatin (Sigma) diluted in CMF-PBS) is added and incubated for 30 min. at 37° C. Plates are washed three times with PBS with 0.05% Tween 20 (PBST) and 50 μl culture supernatant is added. After incubation at 37° C. for 30 minutes, and washing as above, 50 μl of horseradish peroxidase conjugated goat anti-mouse IgG(Fc) (Jackson ImmunoResearch, West Grove, Pa.) diluted 1:3500 in PBST is added. Plates are incubated as above, washed four times with PBST, and 100 μl substrate, consisting of 1 mg/ml o-phenylene diamine (Sigma) and 0.1 μl/ml 30% H₂O₂ in 100 mM Citrate, pH 4.5, are added. The color reaction is stopped after 5 minutes with the addition of 50 μl of 15% H2SO4. A490 is read on a plate reader (Dynatech).

Selected fusion wells are cloned twice by dilution into 96-well plates and visual scoring of the number of colonies/well after 5 days. The monoclonal antibodies produced by hybridomas are isotyped using the Isostrip system (Boehringer Mannheim, Indianapolis, Ind.).

In addition to the production of monoclonal antibodies, techniques developed for the production of “chimeric antibodies”, the splicing of mouse antibody genes to human antibody genes to obtain a molecule with appropriate antigen specificity and biological activity can be used (Morrison et al., Proc Natl Acad Sci 81: 6851-6855, 1984; Neuberger et al., Nature 312: 604-608, 1984; Takeda et al., Nature 314: 452-454; 1985). Alternatively, techniques described for the production of single chain antibodies (U.S. Pat. No. 4,946,778) can be adapted to produce VEGFR-3-specific single chain antibodies.

Antibody fragments which contain the idiotype of the molecule may be generated by known techniques. For example, such fragments include but are not limited to: the F(ab′)2 fragment which may be produced by pepsin digestion of the antibody molecule; the Fab′ fragments which may be generated by reducing the disulfide bridges of the F(ab′)2 fragment, and the two Fab fragments which may be generated by treating the antibody molecule with papain and a reducing agent.

Antibodies to VEGFR-3 may be used in the qualitative and quantitative detection of mature VEGFR-3 and VEGFR-3 precursor and subcomponent forms, in the affinity purification of VEGFR-3 polypeptides, and in the elucidation of VEGFR-3 biosynthesis, metabolism and function. Detection of VEGFR-3 tyrosine kinase activity may be used as an enzymatic means of generating and amplifying a VEGFR-3 specific signal in such assays. Antibodies to VEGFR-3 may also be useful as diagnostic and therapeutic agents.

Non-human antibodies may be humanized by any methods known in the art. A preferred “humanized antibody” has a human constant region, while the variable region, or at least a CDR, of the antibody is derived from a non-human species. Methods for humanizing non-human antibodies are well known in the art. (see U.S. Pat. Nos. 5,585,089, and 5,693,762). Generally, a humanized antibody has one or more amino acid residues introduced into its framework region from a source which is non-human. Humanization can be performed, for example, using methods described Jones et al. [Nature 321: 522-525, (1986)], Riechmann et al., [Nature, 332: 323-327, (1988)] and Verhoeyen et al. [Science 239:1534-1536, (1988)], by substituting at least a portion of a rodent complementarity-determining region (CDRs) for the corresponding regions of a human antibody. Numerous techniques for preparing engineered antibodies are described, e.g., in Owens and Young, J. Immunol. Meth., 168:149-165 (1994). Further changes can then be introduced into the antibody framework to modulate affinity or immunogenicity.

In an alternative embodiment, rapid, large-scale recombinant methods for generating antibodies may be employed, such as phage display [Hoogenboom et al., J. Mol. Biol. 227: 381 (1991); Marks et al., J. Mol. Biol. 222: 581, (1991)] or ribosome display methods, optionally followed by affinity maturation [see, e.g., Ouwehand et al., Vox Sang 74(Suppl 2):223-232 (1998); Rader et al., Proc Natl Acad Sci USA 95:8910-8915 (1998); Dall'Acqua et al., Curr Opin Struct Biol 8:443-450 (1998)]. Phage-display processes mimic immune selection through the display of antibody repertoires on the surface of filamentous bacteriophage and subsequent selection of phage by their binding to an antigen of choice. One such technique is described in WO Publication No. 99/10494, which describes the isolation of high affinity and functional agonistic antibodies for MPL and msk receptors using such an approach.

Monoclonal antibodies against VEGFR-3 may be coupled either covalently or noncovalently to a suitable supramagnetic, paramagnetic, electron-dense, echogenic or radioactive agent to produce a targeted imaging agent. Antibody fragments generated by proteolysis or chemical treatments or molecules produced by using the epitope binding domains of the monoclonal antibodies could be substituted for the intact antibody. This imaging agent would then serve as a contrast reagent for X-ray, magnetic resonance, sonographic or scintigraphic imaging of the human body for diagnostic purposes.

Anti-VEGFR-3 antibodies and antigen binding fragments thereof may be used to diagnose and quantify VEGFR-3 in various contexts. For example, antibodies against various domains of VEGFR-3 may be used as a basis for VEGFR-3 immunoassays or immunohistochemical assessment of VEGFR-3. Tyrosine kinase activity of VEGFR-3 may be useful in these assays as an enzymatic amplification reaction for the generation of a VEGFR-3 signal. Anti-VEGFR-3 antibodies may also be useful in studying the amount of VEGFR-3 on cell surfaces.

Anti-VEGF-C antibodies antigen binding fragments thereof (referred to as the “VEGF-C composition” or “anti-VEGF-C composition”) are also contemplated for use as inhibitors of VEGFR-3/VEGF-C interactions. Anti-VEGF-C compositions are generated as above for the VEGFR-3 antibodies. All forms of anti-VEGF-C antibodies are considered for use, including, monoclonal antibodies, polyclonal antibodies, chimeric antibodies, anti-idiotype antibodies such as F(ab)′ and F(ab′)2 fragments and single-chain antibodies.

VEGFR-3-Encoding Nucleic Acid Molecules

Applicants envision a wide variety of uses for the compositions of the present invention, including diagnostic and/or therapeutic uses of VEGFR-3 polypeptides and fragments thereof, VEGFR-3 analogues and derivatives, VEGFR-3-encoding nucleic acid molecules, antisense nucleic acid molecules or short-interfering RNAs, anti-VEGFR-3 antibodies and polypeptides comprising an antigen binding fragment of an anti-VEGFR-3 antibody.

VEGFR-3-encoding nucleic acid molecules or fragments thereof may be used as probes to detect and quantify mRNAs encoding VEGFR-3. Assays which utilize nucleic acid probes to detect sequences comprising all or part of a known gene sequence are well known in the art. VEGFR-3 mRNA levels may indicate emerging and/or existing neoplasias as well as the onset and/or progression of other human diseases. Therefore, assays which can detect and quantify VEGFR-3 mRNA may provide a valuable diagnostic tool.

Anti-sense VEGFR-3 RNA molecules are useful therapeutically to inhibit the translation of VEGFR-3-encoding mRNAs where the therapeutic objective involves a desire to eliminate the presence of VEGFR-3 or to downregulate its levels. VEGFR-3 anti-sense RNA, for example, could be useful as a VEGFR-3 antagonizing agent in the treatment of diseases in which VEGFR-3 is involved as a causative agent, for example due to its overexpression.

Additionally, VEGFR-3 anti-sense RNAs are useful in elucidating VEGFR-3 functional mechanisms. VEGFR-3-encoding nucleic acid molecules may be used for the production of recombinant VEGFR-3 proteins and related molecules as separately discussed in this application.

An antisense nucleic acid comprises a nucleotide sequence that is complementary to a “sense” nucleic acid encoding a protein (e.g., complementary to the coding strand of a double-stranded cDNA molecule or complementary to an mRNA sequence). Methods for designing and optimizing antisense nucleotides are described in Lima et al., (J Biol Chem;272:626-38. 1997) and Kurreck et al., (Nucleic Acids Res.;30:1911-8. 2002). In specific aspects, antisense nucleic acid molecules are provided that comprise a sequence complementary to at least about 10, 25, 50, 100, 250 or 500 nucleotides or an entire VEGFR-3 coding strand, or to only a portion thereof. Nucleic acid molecules encoding fragments, homologs, derivatives and analogs of a VEGFR-3 or antisense nucleic acids complementary to a VEGFR-3 nucleic acid sequence of are additionally provided.

In one embodiment, an antisense nucleic acid molecule is antisense to a “coding region” of the coding strand of a nucleotide sequence encoding a VEGFR-3 protein. The term “coding region” refers to the region of the nucleotide sequence comprising codons which are translated into amino acid residues. In another embodiment, the antisense nucleic acid molecule is antisense to a “conceding region” of the coding strand of a nucleotide sequence encoding the VEGFR-3. The term “conceding region” refers to 5′ and 3′ sequences which flank the coding region that are not translated into amino acids (i.e., also referred to as 5′ and 3′ untranslated regions).

Antisense nucleic acids of the invention can be designed according to the rules of Watson and Crick or Hoogsteen base pairing. The antisense nucleic acid molecule can be complementary to the entire coding region of VEGFR-3 mRNA, but more preferably is an oligonucleotide that is antisense to only a portion of the coding or noncoding region of VEGFR-3 mRNA. An antisense oligonucleotide can be, for example, about 5, 10, 15, 20, 25, 30, 35, 40, 45, or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation.

The antisense nucleic acid molecules of the invention are typically administered to a subject or generated in situ such that they hybridize with or bind to cellular mRNA and/or genomic DNA encoding VEGFR-3 to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix.

An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). Additional routes of antisense therapy may be used in the invention, e.g. topical admisitration, transdermal administration [reviewed by Brand in Curr. Opin. Mol. Ther. 3:244-8. 2001] antisense administration using nanoparticulate systems [Lambert et al., Adv. Drug. Deliv. Rev. 47:99-112. 2001], or administration of antisense nucleotides conjugated with peptide [Juliano et al., Curr. Opin. Mol. Ther. 2:297-303. 2000].

In still another embodiment, RNA of the invention can be used for induction of RNA interference (RNAi), using double stranded (dsRNA) (Fire et al., Nature 391: 806-811. 1998) or short-interfering RNA (siRNA) sequences (Yu et al., Proc Natl Acad Sci USA. 99:6047-52. 2002). “RNAi” is the process by which dsRNA induces homology-dependent degradation of complimentary mRNA. In one embodiment, a nucleic acid molecule of the invention is hybridized by complementary base pairing with a “sense” ribonucleic acid of the invention to form the double stranded RNA. The dsRNA antisense and sense nucleic acid molecules are provided that correspond to at least about 20, 25, 50, 100, 250 or 500 nucleotides or an entire VEGFR-3 coding strand, or to only a portion thereof. In an alternative embodiment, the siRNAs are 30 nucleotides or less in length, and more preferably 21- to 23-nucleotides, with characteristic 2- to 3-nucleotide 3′-overhanging ends, which are generated by ribonuclease III cleavage from longer dsRNAs. See e.g. Tuschl T. (Nat Biotechnol. 20:446-48. 2002).

Intracellular transcription of small RNA molecules can be achieved by cloning the siRNA templates into RNA polymerase III (Pol III) transcription units, which normally encode the small nuclear RNA (snRNA) U6 or the human RNAse P RNA H1. Two approaches can be used to express siRNAs: in one embodiment, sense and antisense strands constituting the siRNA duplex are transcribed by individual promoters (Lee, et al. Nat. Biotechnol. 20, 500-505. 2002); in an alternative embodiment, siRNAs are expressed as stem-loop hairpin RNA structures that give rise to siRNAs after intracellular processing (Brummelkamp et al. Science 296:550-553. 2002) (herein incorporated by reference).

The dsRNA/siRNA is most commonly administered by annealing sense and antisense RNA strands in vitro before delivery to the organism. In an alternate embodiment, RNAi may be carried out by administering sense and antisense nucleic acids of the invention in the same solution without annealing prior to administration, and may even be performed by administering the nucleic acids in separate vehicles within a very close timeframe. Nucleic acid molecules encoding fragments, homologs, derivatives and analogs of a VEGFR-3 or antisense nucleic acids complementary to a VEGFR-3 nucleic acid sequence are additionally provided.

The invention further contemplates use of the polynucleotides of the invention for gene therapy or in recombinant expression vectors which produce VEGFR-3 polynucleotides or polypeptides of the invention that can regulate activity of VEGFR-3, and are useful in therapy of chronic arthridites characterized by elevated levels of VEGF-C polypeptides. Delivery of a functional gene encoding polypeptides of the invention to appropriate cells is effected ex vivo, in situ, or in vivo by use of vectors, including viral vectors (e.g., adenovirus, adeno-associated virus, or a retrovirus), or ex vivo by use of physical DNA transfer methods (e.g., liposomes or chemical treatments). See, for example, Anderson, Nature, supplement to vol. 392, no. 6679, pp.25-20 (1998). For additional reviews of gene therapy technology see Friedmann, (Science, 244: 1275-1281. 1989); Verma, (Scientific American: 263:68-72, 81-84. 1990); and Miller, (Nature, 357: 455-460. 1992). Introduction of any one of the VEGFR-3 nucleotides of the present invention or a gene encoding VEGFR-3 polypeptides of the invention can also be accomplished with extrachromosomal substrates (transient expression) or artificial chromosomes (stable expression). Cells may also be cultured ex vivo in the presence of proteins of the present invention in order to proliferate or to produce a desired effect on or activity in such cells. In another embodiment, cells comprising vectors expressing VEGFR-3 polynucleotides or polypeptides of the invention may be cultured ex vivo and administered to an individual in need of treatment for chronic arthridites VEGFR-3 polypeptide or polynucleotide treated cells or proliferating cells carrying VEGFR-3 expression vectors can then be introduced in vivo for therapeutic purposes.

Further contemplated are recombinant expression vectors comprising at least a fragment of the polynucleotides set forth above and host cells or organisms transformed with these expression vectors. Useful vectors include plasmids, cosmids, lambda phage derivatives, phagemids, and the like, that are well known in the art. Accordingly, the invention also provides a vector including a polynucleotide of the invention and a host cell containing the polynucleotide. In general, the vector contains an origin of replication functional in at least one organism, convenient restriction endonuclease sites, and a selectable marker for the host cell. Vectors according to the invention include expression vectors, replication vectors, probe generation vectors, and sequencing vectors. A host cell according to the invention can be a prokaryotic or eukaryotic cell and can be a unicellular organism or part of a multicellular organism.

Large numbers of suitable vectors and promoters are known to those of skill in the art and are commercially available for generating the recombinant constructs of the present invention. The following vectors are provided by way of example. Bacterial: pBs, phagescript, PsiX174, pBluescript SK, pBs KS, pNH8a, pNH16a, pNH18a, pNH46a (Stratagene); pTrc99A, pKK223-3, pKK233-3, pDR540, pRIT5 (Pharmacia). Eukaryotic: pWLneo, pSV2cat, pOG44, PXTI, pSG (Stratagene) pSVK3, pBPV, pMSG, and pSVL (Pharmacia). Use of mammalian expression vectors is exemplified above in the description of the FLT4-Ig polypeptide, while use of adenoviral vectors is exemplified in Example 5, infra.

Formulation of Pharmaceutical Compounds

The VEGFR-3 inhibitor and anti-VEGF-C compositions are preferably administered in composition with one or more pharmaceutically acceptable carriers. Pharmaceutical carriers used in the invention include pharmaceutically-acceptable salts, particularly where a basic or acidic group is present in a compound. For example, when an acidic substituent, such as —COOH, is present, the ammonium, sodium, potassium, calcium and the like salts, are contemplated as preferred embodiments for administration to a biological host. When a basic group (such as amino or a basic heteroaryl radical, such as pyridyl) is present, then an acidic salt, such as hydrochloride, hydrobromide, acetate, maleate, pamoate, phosphate, methanesulfonate, p-toluenesulfonate, and the like, is contemplated as a preferred form for administration to a biological host.

Similarly, where an acid group is present, then pharmaceutically acceptable esters of the compound (e.g., methyl, tert-butyl, pivaloyloxymethyl, succinyl, and the like) are contemplated as preferred forms of the compounds, such esters being known in the art for modifying solubility and/or hydrolysis characteristics for use as sustained release or prodrug formulations.

In addition, some compounds may form solvates with water or common organic solvents. Such solvates are contemplated as well.

Pharmaceutical anti-VEGF-C and VEGFR-3 inhibitor compositions can be used directly to practice materials and methods of the invention, but in preferred embodiments, the compounds are formulated with pharmaceutically acceptable diluents, adjuvants, excipients, or carriers. The phrase “pharmaceutically or pharmacologically acceptable” refer to molecular entities and compositions that do not produce adverse, allergic, or other untoward reactions when administered to an animal or a human, e.g., orally, topically, transdermally, parenterally, by inhalation spray, vaginally, rectally, or by intracranial injection. (The term parenteral as used herein includes subcutaneous injections, intravenous, intramuscular, intracisternal injection, or infusion techniques. Administration by intravenous, intradermal, intramusclar, intramammary, intraperitoneal, intrathecal, retrobulbar, intrapulmonary injection and or surgical implantation at a particular site is contemplated as well.) Generally, this will also entail preparing compositions that are essentially free of pyrogens, as well as other impurities that could be harmful to humans or animals. The term “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art.

The pharmaceutical compositions containing the anti-VEGF-C or VEGFR-3 inhibitors described above may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any known method, and such compositions may contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452, and 4,265,874 to form osmotic therapeutic tablets for controlled release.

Formulations for oral use may also be presented as hard gelatin capsules wherein the active ingredient is mixed with an inert solid diluent, for example, calcium carbonate, calcium phosphate or kaolin, or as soft gelating capsules wherein the active ingredient is mixed with water or an oil medium, for example peanut oil, liquid paraffin, or olive oil.

Aqueous suspensions may contain the active compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compositions may also be in the form of suppositories for rectal administration of the PTPase modulating compound. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols, for example.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Administration and Dosing

Some methods of the invention include a step of VEGFR-3 inhibitor administration to a human or animal. Polypeptide or polynucleotide VEGFR-3 inhibitors may be administered in any suitable manner using an appropriate pharmaceutically-acceptable vehicle, e.g., a pharmaceutically-acceptable diluent, adjuvant, excipient or carrier. The composition to be administered according to methods of the invention preferably comprises (in addition to the polypeptide, polynucleotide or vector) a pharmaceutically-acceptable carrier solution such as water, saline, phosphate-buffered saline, glucose, or other carriers conventionally used to deliver therapeutics or imaging agents.

The “administering” that is performed according to the present invention may be performed using any medically-accepted means for introducing a therapeutic directly or indirectly into a mammalian subject, including but not limited to injections (e.g., intravenous, intramuscular, intra-articular, subcutaneous, or catheter); oral ingestion; intranasal or topical administration; and the like. The therapeutic composition may be delivered to the patient at multiple sites. The multiple administrations may be rendered simultaneously or may be administered over a period of several hours. In certain cases it may be beneficial to provide a continuous flow of the therapeutic composition. Additional therapy may be administered on a period basis, for example, daily, weekly or monthly.

Polypeptides and polynucleotides for administration may be formulated with uptake or absorption enhancers to increase their efficacy. Such enhancer include for example, salicylate, glycocholate/linoleate, glycholate, aprotinin, bacitracin, SDS caprate and the like. See, e.g., Fix (J. Pharm. Sci., 85:1282-1285, 1996) and Oliyai and Stella (Ann. Rev. Pharmacol. Toxicol., 32:521-544, 1993).

The amounts of peptides in a given dosage will vary according to the size of the individual to whom the therapy is being administered as well as the characteristics of the disorder being treated. In exemplary treatments, it may be necessary to administer about 50 mg/day, 75 mg/day, 100 mg/day, 150 mg/day, 200 mg/day, 250 mg/day. These concentrations may be administered as a single dosage form or as multiple doses. Standard dose-response studies, first in animal models and then in clinical testing, reveal optimal dosages for particular disease states and patient populations.

It will also be apparent that dosing should be modified if traditional therapeutics are administered in combination with therapeutics of the invention. For example, treatment of chronic arthridites using traditional anti-inflammatory or other arthritis directed therapeutics, in combination with methods of the invention, is contemplated.

Medical Imaging

Anti-VEGF-C antibodies or fragments thereof that bind to VEGF-C ligand are useful in medical imaging, e.g., imaging the site of inflammation and other sites having VEGF-C molecules. See, e.g., Kunkel et al., U.S. Pat. No. 5,413,778. Such methods involve chemical attachment of a labeling agent, administration of the labeled VEGF-C binding polypeptide to a subject in a pharmaceutically acceptable carrier, and imaging the labeled VEGF-C binding polypeptide in vivo at the target site. The above method is used to image VEGFR-3 polypeptides in the same manner.

The potential efficacy of VEGFR-3 inhibitors, e.g. fragments of an anti-VEGFR-3 antibody, polypeptides comprising a soluble VEGFR-3 fragment, extracellular domain fragments of human VEGFR-3, and anti-VEGF-C polypeptides, to ameliorate symptoms associated with rheumatoid arthritis or chronic arthridites is demonstrated, e.g., using procedures such as those described in the following examples, some of which are prophetic The examples assist in further describing the invention, but are not intended in any way to limit the scope of the invention.

### EXAMPLE 1

### VEGF-C is Increased and VEGF-D Decreased in the Rheumatoid Synovial Lining

In order to assess the levels of VEGF-C and -D ligand in rheumatoid arthritis (RA) patients, synovial membrane samples were collected with the permission of the local ethics committee and with the patients' consent during joint replacement surgery or arthroscopic procedures. Sections were stained with hematoxylin and eosin and reviewed by an experienced histopathologist. Patient records were reviewed to ensure that all the patients met the disease criteria (Arnett et al., Arthritis Rheum. 31:315-24. 1988; Dougados et al., Arthritis Rheum. 34:1218-27. 1991). RA patients were undergoing various treatment regimens with common therapeutics which include sulfasalazopyrin, methotrexate, prednisolone, cyclosporine, hydroxychloroquine, sodium aurothiomalate and NSAIDs.

For cryosections staining, 8 cases of RA (from hip (4), metacarpophalangeal (1), knee (2) and elbow (2) joints), 4 cases of ankylosing spondylitis (AS) (from hip (1), knee(2) and shoulder joints (1)) and 12 controls from trauma knee joints were studied. The tissues were snap frozen in liquid nitrogen and embedded in OCT-compound (Sakura, Torrance, Calif.). Adjacent 5 μm tissue sections were air-dried and fixed in cold acetone for 10 minutes. The sections were incubated with the appropriate blocking serum (5% normal horse or goat serum) and with the appropriate primary antibody overnight at 4° C. Primary antibodies against VEGFs and their receptors were as follows: VEGF-C (pAB 882, Joukov et al, EMBO J. 15:290-98. 1996; VEGF-D (pAb 749-1AP or 78912.11, R & D Systems, Minneapolis, Minn.); VEGFR-2 (KDR-1, Simon et al., 1996) and VEGFR-3 (9D9F9, Valtola et al., Am. J. Pathol. 154:1381-90. 1999). Other antibodies used were: monoclonal anti-CD31 Ab (1:300, DAKO Immunoglobulins, Glostrup, Denmark), monoclonal PAL-E Ab (36) (1:400, Monosan, Uden, the Netherlands), monoclonal anti-laminin Ab (1:2000, clone LAM-89, Sigma, St. Louis, Mo., USA), monoclonal anti-smooth muscle-actin Ab (1:10,000, clone 1A4, Sigma), monoclonal anti-IFN-Ab (1:200, BD PharMingen, San Diego, Calif., USA). polyclonal rabbit antiserum to IL-1 (1:500, Genzyme, Cambridge, Mass., USA), polyclonal rabbit antiserum to IL-6 (1:1000, Genzyme) and a polyclonal rabbit antiserum to TNF-α (1:500, Monosan). A 30-minute incubation with the appropriate secondary antibody (biotinylated anti-mouse or anti-rabbit antibodies) was followed with a 60 minute incubation with Vectastain Elite avidin-biotin complex (ABC)/HRP kit (Vector Laboratories, Burlingame, Calif., USA) and by development of peroxidase activity with 3-amino-9-ethyl carbazole (Sigma) or 3,3-diaminobenzidine tetrahydrochloride (Sigma). The slides were briefly counterstained with hematoxylin and mounted in Aquamount (BDH Laboratories, Dorset, England). Negative staining controls were done by omitting the primary Ab or by using irrelevant primary Abs of the same isotype (mouse IgG1 or rabbit IgG). Specificity controls of VEGF-C Abs were done using an antigen preabsorption test with a 40-fold molar excess of the purified immunogen. Immunoreactivity and specificity of the anti-VEGF-D antibodies was verified by immunofluorescence in 293EBNA cells transiently transfected with VEGF-D. Staining intensity was graded as follows by blinded histopathological assessment of the entire synovial sample area: −, no staining; + weak staining and/or few positive cells; ++, moderate staining and/moderate numbers of positive cells; +++, strong staining and/or numerous positive cells.

Immunostaining of joint tissue revealed weak (+) VEGF-C expression in control synovial lining and fibroblast-like stromal cells. Control samples also stained moderately (++) for VEGF-D. Although VEGF-C staining was present in the thin, single-cell synovial lining of control samples, it was stronger (+++) and present in many more cells in the thickened lining cell layer in RA and AS patients. In contrast, and unlike in the healthy controls, there was only weak (+) or no VEGF-D staining in the RA or AS samples. VEGF-C staining was specific as it was blocked with a 40-fold molar excess of the immunogenic VEGF-C peptide.

### EXAMPLE 2

### VEGF-C and Receptors in Synovial Membrane

To assess whether VEGF-C ligand was present in the synovial membrane as well as the synovial lining, tissue samples were stained as described above with anti-VEGF-C/D and anti-VEGFR antibodies and blood vessel markers, wherein the distinction between pericytes, smooth muscle cells (SMC) and endothelial cells was based on staining using antibodies against smooth muscle actin (SMA), PAL-E and laminin, respectively.

VEGF-C and VEGF-D were localized to blood vessel pericytes and SMCs in all samples. VEGFR-2 was detected in the endothelial cells of the same vessels, suggesting that the ligands have a paracrine mode of action. VEGF-C and VEGF-D were also expressed in stromal fibroblasts and macrophages in inflamed synovial tissue. VEGF-C staining was often found adjacent to its receptor VEGFR-3 in subsynovial capillaries and venules, but such a co-localization was much less common in the stromal vessels.

The proximity of VEGF-C to its receptors VEGFR-2 and VEGFR-3 in synovial blood vessel endothelium indicates a role for the VEGFR-2/3:VEGF-C interaction in angiogenesis associated with the progression of rheumatoid arthritis and chronic arthridites.

Staining of synovial tissue for inflammatory cytokines also demonstrated that VEGF-C co-localized partially with the inflammatory cytokines IL-1, IL-6 and TNF-α in RA synovial lining cell layer. There was little VEGF-D or IFN-γ staining present in the rheumatoid synovial lining.

### EXAMPLE 3

### Expression of VEGFR-3 in Synovial Blood Vessels

The increased expression of VEGF-C in arthritic patients caused us to investigate whether the level of the VEGF-C receptor VEGFR-3 is also increased in these individuals. Staining of tissue samples was carried out as described in Example 1 to determine the expression level and location of VEGF-C receptors in synovial vessels.

The lymphatic endothelial receptor VEGFR-3 was detected in most of the blood vessels in control and AS and RA samples, primarily in the sublining capillaries and venules. In particular, in the sublining capillaries responsible for fluid filtration VEGFR-3 was expressed in the PAL-E positive blood vascular endothelial cells. Only a few vessels were VEGFR-3 positive and PAL-E negative, suggesting that they were true synovial lymphatic vessels. VEGFR-3 staining of the lymphatic vessels was more intense than that of the blood vessels. Although it was difficult to count the number of lymphatic vessels due to VEGFR-3 on sublining blood vessel capillary endothelium, the ratio of lymphatic vessels to blood vessels was clearly lower in RA samples than in controls, mainly as a result of increased vascularity.

Although the exact role of VEGFR-3 in the synovial lining remains to be determined, the increased vascularity resulting from increased VEGF-C/VEGFR-3 interaction may contribute to inflammation, pannus formation, bone and cartilage destruction and disease progression in RA. Interestingly, most disease-modifying, anti-rheumatic drugs have anti-angiogenic effects (Walsh D A. Rheumatology. 38:103-12. 1999) and some of them, such as corticosteroids, may block the induction of VEGF-C by the inflammatory cytokines (Ristimaki et al. J Biol. Chem. 273:8413-8. 1998). Additionally, synovial membrane contains fenestrated blood vessels expressing VEGFR-3 which are involved in the nutrition of the avascular hyaline articular cartilage. Thus, VEGFR-3 may be involved in the maintenance of fenestrations and in the formation of the synovial fluid.

### EXAMPLE 4

### Treatment of Rheumatoid Arthritis with VEGFR-3 Inhibitor Compositions

Previous experiments have demonstrated that soluble VEGFR-3 inhibited the activity of VEGF-C in vivo and in vitro. A fusion protein consisting of the first three Ig-homology domains of VEGFR-3 and IgG Fe bound VEGF-C and VEGF-D with the same efficiency as the full-length extracellular domain and inhibited VEGF-C-induced VEGFR-3 phosphorylation. In vivo, the VEGFR-3-Ig fusion protein was expressed under the control of K14 promoter, which directs transgene expression to the basal epidermal cells of the skin. VEGFR-3-Ig expression is detected in mice by northern blotting of skin RNA and by western blotting of protein extracts from the skin. When the skin sections were stained for markers of the lymphatic endothelium, VEGFR-3 (Jussila et al., Cancer Res. 58:1599-1604, 1998; Kubo et al., Blood, 96 546-553, 2000) and LYVE-1 (Banerji et al., J Cell Biol, 144: 789-801, 1999), no lymphatic vessels were observed in the transgenic mice, even though lymphatic vessels were stained in the skin of control mice. These results indictate that VEGFR-3 inhibitors are effective blockers of lymphangiogenesis.

In order to assess the ability of VEGFR-3 inhibitory compositions to modulate the progression of RA, a mouse model of RA is employed.

Collagen induced arthritis (CIA), a model of human RA, is induced in 8-12 week old DBA/1 mice by immunization with chick type II collagen in complete Freund's adjuvant as described by Campbell et al (J. Clin. Invest. 107:1519-1527. 2001). Briefly, chick type II collagen dissolved in 10 mM acetic acid at 2 mg/ml is emulsified in an equal volume of adjuvant containing 5 mg/ml heat-killed Mycobacterium tuberculosis (strain H37Ra). Arthritis is then induced by injecting mice intradermally at several sites into the base of the tail with 100 μl emulsion at days 0 and 21. Animals are assessed for arthritis in the paws (erythema and swelling of limbs) 2-3 times per week as outlined in Campbell et al (J. Clin. Invest. 05:1799-1806. 2000).

Clinical signs of disease arise between 21 and 30 days in mice induced with type II collagen. To assess the efficacy of VEGFR-3 inhibitory treatment immunized mice are treated with compositions comprising a VEGFR-3 inhibitor over a varying range of doses deemed appropriate by initial dosing studies (e.g. 0.25 to 3.0 mg/kg) beginning in different stages of disease progression. Animals are treated intraperitoneally, intravenously, intra-articularly or subcutaneously with the VEGFR-3 compositions for 7 days beginning, for example, on day 0, day 7, day 14 or day 21 pre-disease onset, or beginning treatment directly after detection of arthritis in the joints of subject animals.

From the first appearance of clinical signs of CIA, joint swelling is measured daily with precision calipers (using in the paw initially showing signs of disease). Swelling in animals treated with VEGFR-3 inhibitory compositions is compared to swelling in mice treated with control protein and animals treated with the inhibitory compositions but immunized with control protein.

The severity of arthritis is evaluated based on an arthritis index. See U.S. Pat. No. 5,888,510. Briefly, the evaluation is based on a 4 point scale for each limb, for a total of 16 points per animal. The evaluation standard is as follows: 0.5, erythema observed at one site of joint; 1, erythema observed at two sites of joint, or redness but no swelling of dorsa; 2, moderate swelling observed; 3, severe swelling of pedal dorsa, but not reaching all of the digits; 4, severe swelling of pedal dorsa and digits. For all evaluations, a representative population of animals is chosen for each analysis.

In addition to swelling, the progression of CIA in subject animals is assessed by histological means. Synovial membrane samples from the joints of control and treated animals are isolated and stained with antibodies to VEGF-C and its receptors to analyze the progression of new lymphatic vessel formation. The presence or absence of VEGF-C and VEGFR-3 in lymphatic endothelial cells, vascular endothelial cells, synovial lining cell layers and stromal macrophages is assessed.

The mice are sacrificed over a range of timepoints (i.e. day 7, 14, 21, 28 or 35) after collagen immunization, and the hind legs fixed with 20% formalin. The samples are then subjected to demineralization in an EDTA solution (pH 7.6) and dewatering with alcohol. They are subsequently wrapped in paraffin and cut to 2 μm or 5 μm thick sections. The sections are stained with hematoxylin and eosin or various primary stains, and observed under 125× magnification.

Staining is carried out as described previously. Briefly, 5 μm tissue sections are fixed in acetone for 10 min. and incubated with appropriate blocking serum (5% normal goat serum) and primary antibody [e.g. VEGF-C (pAb 882), VEGF-D (pAb-749-1AP), VEGFR-3 (9D9F9), VEGFR-2 (KDR-1)]. Sections are then incubated with appropriate secondary antibody for 30 min followed by a 60 min incubation with Vectastain Elite biotin-avidin complex-horseradish peroxidase (HRP) kit (Vector, Burlingame, Calif.). Staining intensity (graded by blind assessment of histological sections) is graded in different synovium structures in control and CIA membranes (+++, strong staining; ++, moderate staining; +, weak staining; −, no staining).

Treatment with compositions comprising a VEGFR-3 inhibitor is expected to diminish the levels of VEGF-C and VEGFR-3 detectable in the synovial tissue sections of arthritic animals, and also result in a decrease in the degree of joint swelling measured in treated animals as compared to control animals.

In addition to analyzing the presence of the growth factors and receptors, the effects of VEGFR-3 inhibitors on the general progression and destruction resulting from RA is assessed via histology of cartilage erosion and assessment of the release of cartilage oligomeric matrix protein by ELISA.

### EXAMPLE 5

### Induction of Rheumatoid Arthritis in Mice Constitutively Expressing VEGFR-3-Ig

It has been shown previously that soluble VEGFR-3 binds to VEGF-C equally as efficiently as the non-soluble receptor and subsequently inhibits VEGF-C mediated signaling in vitro (U.S. Ser. No. 09/765,534, herein incorporated by reference). To assess the effects of soluble VEGFR-3 on rheumatoid arthritis, transgenic mice constitutively expressing a soluble VEGFR-3 are made.

Transgenic mice expressing soluble VEGFR-3-Ig constructed as described in U.S. Ser. No. 09/765,534 are used. Briefly, the sequence encoding human VEGFR-3 Ig-homology domains 1-3 was amplified using PCR. The primers employed for this purpose were: 5′-TACAAAGCTTTTCGCCACCATGCAG-3′ (SEQ ID NO:5) and ′5-TACAGGATCCTCATGCACAATGACCTC-3′ (SEQ ID NO:6).

The PCR product was cloned into the pIg-plus vector (Ingenius, R&D Systems) in frame with human IgG1 Fc tail. The VEGFR-3-Ig construct was then transferred into the human keratin-14 promoter-expression vector. The expression cassette fragment was injected into fertilized mouse oocytes of the FVB/NIH and DBAxBalbC hybrid strains to create seven lines of K14-VEGFR-3-Ig mice.

To assess the presence of the VEGFR-3-Ig transgene in the transgenic susceptible strains northern blotting analysis is used. Briefly, 10 μg of total RNA extracted from skin in 1% agarose was subjected to electrophoresis, transferred to nylon filters (Nytran), hybridized with the corresponding [³²P]-labeled cDNA probes and exposed autoradiography. For western blotting, skin biopsies are homogenized into the lysis buffer (20 mM Tris, pH 7.6, 1 mM EDTA, 50 mM NaCl, 50 mM NaF, 1% Triton-X100) supplemented with 1 mM PMSF, 1 mU/ml approtinin, 1 mM Na3VO4 and 10 μg/ml leupeptin. The Ig-fusion proteins are precipitated from 1 mg of total protein and separated in SDS-PAGE, transferred to nitrocellulose and detected using the horseradish peroxidase conjugated rabbit antibodies against human IgG (DAKO, Carpinteria, Calif.) and the enhanced chemiluminescence detection system.

Analysis of lymphangiogenesis in these mice indicates that VEGFR-3-Ig expression suppressed lymphangiogenesis in the ear skin. Additionally, VEGFR-3-Ig expression also induced regression of the already-formed lymphatics. Thus, inhibition of VEGF-C and/or VEGF-D binding to VEGFR-3 during development leads to apoptosis of the lymphatic endothelial cells and to the disruption of the lymphatic network, which indicates that continuous VEGFR-3 signaling is required for the survival of the lymphatic endothelial cells.

In young VEGFR-3-Ig transgenic mice, several internal organs were almost completely devoid of lymphatic vessels, but they regrew in adult mice, although into an abnormal pattern in some organs. The growth and maintenance of lymphatic vasculature can therefore be reactivated in adult organs.

Transgenic mice expressing soluble VEGFR-3-Ig constructed as above are crossed onto the DBA/1 background or other murine genetic backgrounds susceptible to induction of collagen induced arthritis.

To assess the effects of soluble VEGFR-3 on development of rheumatoid arthritis, adult VEGFR-3-Ig/DBA/1 transgenic mice are induced with collagen induced arthritis as described previously. The severity of arthritis is evaluated based on an arthritis index as described above, and the progression of CIA in subject animals is assessed by histological means. Using protocols described above, synovial membrane samples from the joints of control and VEGFR-3 transgenic animals are isolated and stained with antibodies to VEGF-C and its receptors to analyze the progression of new lymphatic vessel formation. The presence or absence of VEGF-C and VEGFR-3 in lymphatic endothelial cells, vascular endothelial cells, synovial lining cell layers and stromal macrophages of arthritic VEGFR-3-Ig/DBA/1 mice is assessed.

A decrease of the lymphatic vasculature in and around synovial sites in collagen induced arthritic VEGFR-3-Ig transgenic mice may result in a decrease in the cellular infiltrate into those sites which contribute to the inflammatory environment and joint swelling common in arthritic diseases. A result of this nature indicates that VEGFR-3 inhibition is an effective method for ameliorating symptoms associated with rheumatoid arthritis and chronic arthridites.

Soluble VEGFR-3 is a potent and specific inhibitor of lymphangiogenesis in vivo. In addition to the VEGFR-3 construct above, the soluble VEGFR-3 construct containing an extracellular fragment of VEGFR-3 may be a fragment of VEGFR-3 which comprises more or less of the wild-type sequence of VEGFR-3. For example, the soluble peptide also may comprise VEGFR-3 domains IgI to IgIII in any combination with one or more of the domains selected from the group consisting of IgIV, IgV, IgVI and IgVII.

The soluble VEGFR-3-Ig protein is administered into the joint space of CIA induced DBA mice via intra-articular injection of an appropriate dose of fusion protein, determined prior to the start of the experiment by dose curve analysis. The effects of soluble VEGFR-3-Ig administration on onset and progression of rheumatoid arthritis is then assessed as described above, via joint swelling analyses and histological assessment of cellular infiltrate and lymphatic vasculature at the synovial site.

The effects of VEGFR-3 on the progression of rheumatoid arthritis are also assessed using gene therapy techniques. Adenovial vectors expressing soluble VEGFR-3-Ig as in Karpanen et al., Cancer Research 61: 1786-90. 2001, (incorporated herein by reference) are used. Briefly, the cDNA coding for the VEGFR-3-Ig fusion protein was subcloned into the pAdCMV plasmid, constructed by subcloning the human cytomegalovirus immediate-early promoter, the multiple cloning site, and the bovine growth hormone gene polyadenylation signal from the pcDNA3 (Invitrogen) into the pAdBglII vector, and the adenoviruses were produced as described previously (Laitinen et al. Hum. Gene Ther., 9: 1481-1486, 1998).

DBA mice are induced with collagen induced arthritis as described above and the VEGFR-3-Ig (AdR3-Ig) or LacZ control (Laitinen et al, supra) adenoviruses are injected at varying concentrations (ranging from 5×10−6 to 5×10−9 plaque forming units (pfu) into arthritis susceptible mice. The adenoviral vectors are administered either i.v., i.p., sub-cutaneously or intra-articularly (e.g. at the knee-joint). AdR3-Ig is administered before the onset of clinical signs of RA, at approximately 25 days post induction. Treated and control animals are monitored for onset of disease as above and are sacrificed at varying times after disease onset (d3, d7, d10, d14 post onset) for histological assessment of cellular infiltrate, VEGF-C and VEGFR-3 expression and lymphangiogenesis. In another embodiment, the adenoviral vectors are administered at varying times during the course of disease, including day 0, day 1, day 3, day 7, day 14 post induction or at times after the onset of disease to investigate the inhibition of VEGFR-3 on the progression and amelioration of acute disease. It is further contemplated that the adenoviral vector is administered multiple times on any of the days after induction of arthritis as exemplified above, to maintain a constant level of soluble VEGFR-3-Ig protein at the synovial site.

### EXAMPLE 6

### Treatment of Human Rheumatoid Arthritis or Chronic Arthridites with VEGFR-3 Inhibitor Compositions

Human patients with rheumatoid arthritis or chronic arthridites are assessed for improvement in arthritic symptoms as described in U.S. Pat. No. 5,858,446 (Weiner et al) after treatment with VEGFR-3 inhibitory compounds alone or VEGFR-3 compounds in conjunction with known arthritis treatments. The patient's arthritic state is measured utilizing a combination of several different criteria of acute arthritis or rheumatoid arthritis as set out by the American Rheumatology Association (Arnett et al, Arthritis Rheum. 31:315-24. 1988) such as morning stiffness, arthritis in several joint areas, arthritis of hand joints, symmetric arthritis, rheumatoid nodules, serum rheumatoid factor, and radiographic changes (erosions or decalcification). Subjective pain, gross anatomical observations, timing of physical acts and subjective well-being as described by the patient are also considered. Gross anatomical observations included AM stiffness, grip strength and number of swollen joints and are made during monthly examinations by a physician of the arthritic joints before and during the VEGFR-3 inhibitor or combination treatment as compared with the same joints prior to treatment.

Data measuring subjective pain involves applying gentle pressure to each arthritic joint in turn by a physician and being told by the patient whether pain is experienced.

Morning stiffness data is based on the patient's experience and reports on how long it took for their arthritic joints to become physically limber. Additionally, grip strength for each hand is measured at least once a month or more often with a standard mercury sphygmomanometer with the cuff inflated to 20 mm Hg. Additionally, the patients are timed to measure how many seconds are needed to complete a 50-foot walk.

The efficacy of administration of VEGFR-3 inhibitory compositions to humans afflicted with rheumatoid arthritis is evaluated using three different criteria: the Paulus Response Criteria, the American College of Rheumatology Criteria, and the Protocol Response Criteria. According to the Protocol Response Criteria, a positive response is scored when a 30% improvement in tender and swollen joint counts is achieved in a subject.

In the American College of Rheumatology Response Criteria, a positive response is scored when:

a) a 20% improvement in tender and swollen joint counts is achieved and b) there is a 20% improvement in any 3 of the following: (1) patient global score; (2) physician global score; (3) patient pain score; (4) CLINHAQ (clinical health assessment questionnaire); (5) ESR (erythrocyte sedimentation rate).

A positive result is scored in the Paulus Response Criteria when 4 of the following 6 criteria are satisfied: a) a 20% improvement in (1) tender joint score; (2) swollen joint score; (3) duration of morning stiffness (4) ESR; b) a. 40% improvement in (5) physician global score; (6) patient global score.

The protocols required subject examination by a physician of each subject participating in the study at 2, 4, 8, 12, 16, 20, and 24 weeks from the baseline date (date of entry into the study). During each examination the physician evaluates the subject for the criteria included in the Paulus Response, the American College of Rheumatology Response, and the Protocol Response evaluations.

It is understood that, inherent in the invention, any clinically or statistically significant amelioration of any symptom of arthritis resulting from administration of the VEGFR-3 inhibitory compositions, either alone or in conjunction with already known arthritis therapies, is within the scope of the invention. Clinically significant attenuation means perceptible to the patient (as in the case of tenderness or general well-being) and/or to the physician (as in the case of joint swelling). For example, a difference in swelling or tenderness in only one arthritic joint is considered significant.

VEGFR-3 inhibitor compositions can be administered either alone or in combination with existing drugs used for the treatment of arthritic conditions such as NSAIDs, analgesics, glucocorticoids, DMARDs or biologic response modifiers.

Non-steroidal anti-inflammatory treatments (NSAIDs) for arthritic conditions that are given in conjunction with VEGFR-3 inhibitors include common over the counter therapeutics such as ibuprofen, aspirin, and naproxen, and prescription drugs such as Celecoxib (Celebrex) and Rofecoxib (Vioxx).

Analgesics, used to treat pain in arthritic conditions, are used in combination with VEGFR-3 and anti-VEGF-C compositions and include acetaminophen, acetaminophen with codeine, oxycodone (Oxycontin) and other common prescription drugs. VEGFR-3 is also used in conjunction with glucocorticoids which decrease inflammation in the joint such as cortisone, dexamethosone, methylprednisolone, prednisolone, and other prescription glucocorticoids.

VEGFR-3 is used in combination with disease modifying DMARDs which include leflunomide, cyclophosphamide, cyclosporine, and methotrexate to name a few. Biologic response modifiers target specific biologic responses occurring in arthritic conditions. VEGFR-3 is used in conjunction with drugs such as etanercept (Enbrel) and Infliximab (Remicade) and other biologic response modifiers to ameliorate arthritic symptoms in patients with RA and chronic arthridites.

All documents including patents and journal articles that are cited in the summary or detailed description of the invention are hereby incorporated by reference, in their entirety.

While the invention here has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. A method of treating a mammalian subject affected with chronic arthridites comprising the steps of:
a) screening a mammalian subject with symptoms of chronic arthridites for VEGF-C protein expression in a synovial site; and
b) administering to the mammalian subject identified in the screening step as having elevated VEGF-C expression in a synovial site a composition comprising an inhibitor of vascular endothelial growth factor receptor-3 (VEGFR-3 inhibitor) in an amount effective to ameliorate symptoms of chronic arthridites in said patient.

2. A method according to claim 1 wherein the chronic arthridites is rheumatoid arthritis.

3. A method according to claim 1 wherein the mammalian subject is human.

4. A method according to claim 3 wherein the VEGFR-3 inhibitor is selected from the group consisting of a polypeptide comprising a soluble VEGFR-3 fragment that binds to VEGF-C protein, a VEGFR-3 anti-sense polynucleotide or short-interfering RNA (siRNA), an anti-VEGFR-3 antibody, a polypeptide comprising an antigen binding fragment of an anti VEGFR-3 antibody, and an anti-VEGF-C antibody.

5. A method according to claim 3 wherein the VEGFR-3 inhibitor inhibits VEGF-C binding to VEGFR-3.

6. A method according to claim 4 wherein the VEGFR-3 inhibitor comprises a polypeptide comprising an extracellular domain fragment of mammalian VEGFR-3, wherein said fragment binds to VEGF-C protein.

7. A method according to claim 6 wherein the VEGFR-3 fragment is human.

8. A method according to claim 6 wherein the extracellular domain fragment comprises immunoglobulin-like domains 1 through 3 of VEGFR-3.

9. A method according to claim 6 wherein the extracellular domain fragment comprises amino acids 33 to 324 of the human VEGFR-3 amino acid sequence set forth in SEQ. ID NO.: 4.

10. A method according to claim 6 wherein the soluble VEGFR-3 fragment is linked to an immunoglobulin Fe domain.

11. A method according to claim 3 wherein the inhibitor comprises a polypeptide comprising an amino acid sequence comprising at least 90% amino acid identity to amino acids 33 to 324 of human VEGFR-3 set out in SEQ ID NO: 4 and maintains ligand binding activity of human VEGFR-3.

12. A method according to claim 3 wherein the composition further comprises a pharmaceutically acceptable diluent, adjuvant, or carrier medium.

13. A method according to claim 1, wherein the screening step comprises:
(a) obtaining a biological sample from a synovial site of the mammalian subject; and
(b) measuring VEGF-C polypeptide in the biological sample to identify elevated VEGF-C expression.

14. A method according to claim 13 wherein said biological sample comprises synovial tissue.

15. A method according to claim 13 wherein said biological sample comprises synovial fluid.

16. A method according to claim 3 wherein the chronic arthridites is selected from the group consisting of osteoarthritis, Juvenile Arthritis and Ankylosing Spondylosis, HIV-related arthritis and psoriatic arthritis.

17. A method according to claim 2 wherein the screening step comprises
(a) administering to a mammalian subject with symptoms of chronic arthridites a composition comprising an antibody or antibody fragment that specifically binds VEGF-C; and
(b) determining VEGF-C protein expression based on the quantity or distribution of said antibody in the mammalian subject, wherein an elevated level of VEGF-C expression in synovial sites correlates with the presence of chronic arthridites.

18. A method according to claim 17 further comprising, between the administering step and the determining step, the step of obtaining a biological sample of synovial fluid or synovial tissue from said mammalian subject and determining the quantity and distribution of VEGF-C in the biological sample, wherein an elevated level of VEGF-C expression correlates with the presence of chronic arthridites.

19. A method according to claim 17 or 18 wherein said antibody or antibody fragment further comprises a label.

20. A method according to claim 19 wherein said antibody or antibody fragment is coupled to a radioactive label.

21. A method according to claim 19 wherein said antibody or antibody fragment is coupled to a colorimetric label.

22. A method of treating a mammal having chronic arthridites characterized by elevated VEGF-C protein expression at synovial sites, comprising a step of administering to said mammalian organism a composition, said composition comprising a VEGFR-3 inhibitor which inhibits binding between VEGF-C and VEGFR-3 expressed in cells of said organism, thereby inhibiting VEGFR-3 function.

23. A method according to claim 22 wherein the chronic arthridites is rheumatoid arthritis.

24. A method according to claim 22 wherein the mammal is human.

25. A method according to claim 24 comprising a screening step preceding the administering step,
wherein the screening step comprises screening a human with symptoms of chronic arthridites to identify a chronic arthridites characterized by elevated VEGF-C protein expression; and
wherein the administering step comprises administering the composition to a human identified by the screening step as having chronic arthridites characterized by increased VEGF-C protein expression.

26. A method according to claim 24 wherein the VEGFR-3 inhibitor is selected from the group consisting of a polypeptide comprising a soluble VEGFR-3 fragment that binds to VEGF-C protein, a VEGFR-3 anti-sense polynucleotide or siRNA, an anti-VEGFR-3 antibody, a polypeptide comprising an antigen binding fragment of an anti-VEGFR-3 antibody, and an anti-VEGF-C antibody.

27. A method according to claim 26 wherein the VEGFR-3 inhibitor inhibits VEGF-C binding to VEGFR-3.

28. A method according to claim 26, wherein the VEGFR-3 inhibitor comprises a polypeptide comprising an extracellular domain fragment of mammalian VEGFR-3, wherein said fragment binds to VEGF-C protein.

29. A method according to claim 28 wherein the VEGFR-3 fragment is human.

30. A method according to claim 28 wherein the extracellular domain fragment comprises immunoglobulin-like domains 1 through 3 of VEGFR-3.

31. A method according to claim 28 wherein the extracellular domain comprises amino acids 33 to 324 of the human VEGFR-3 amino acid sequence set forth in SEQ. ID NO.: 4.

32. A method according to claim 28 wherein the soluble VEGFR-3 fragment is linked to an immunoglobulin Fc domain.

33. A method according to claim 24 wherein the inhibitor composition comprises a polypeptide comprising an amino acid sequence comprising at least 90% amino acid identity to amino acids 33 to 324 of human VEGFR-3 set out in SEQ ID NO: 4 and maintains ligand binding activity of human VEGFR-3.

34. A method according to claim 24 wherein the composition further comprises a pharmaceutically acceptable diluent, adjuvant, or carrier medium

35. A method according to claim 24 wherein the chronic arthridites is selected from the group consisting of osteoarthritis, Juvenile Arthritis, Ankylosing Spondylosis, HIV-related arthritis and psoriatic arthritis.

36. A method according to claim 1 or 22 wherein the VEGFR-3 inhibitor is administered in combination with a rheumatoid arthritis medication selected from the group consisting of nonsteroidal anti-inflammatory drugs (NSAIDs), analgesics, glucocorticoids, disease-modifying antirheumatic drugs (DMARDs) and biologic response modifiers.

37. A method according to claim 36 wherein the VEGFR-3 inhibitor is a NSAID selected from the group consisting of ibuprofen, naproxen, naproxen sodium, Cox-2 inhibitors and salicylates.

38. A method according to claim 36 wherein the VEGFR-3 inhibitor is an analgesic selected from the group consisting of acetaminophen, oxycodone, tramadol and propoxyphene hydrochloride.

39. A method according to claim 36 wherein the VEGFR-3 inhibitor is a glucocorticoid selected from the group consisting of cortisone, dexamethosone, hydrocortisone, methylprednisolone, prednisolone and prednisone.

40. A method according to claim 36 wherein the VEGFR-3 inhibitor is a biological response modifier selected from the group consisting of etanercept (Enbrel) and infliximab (Remicade).

41. A method according to claim 36 wherein the VEGFR-3 inhibitor is a DMARD selected from the group consisting of auranofin, azathioprine, cyclophosphamide, cyclosporine, methotrexate and penicillamine.

42. A method according to claim 1 or 22 wherein the administering is performed systemically.

43. A method according to claim 1 or 22 wherein the administering is done locally at synovial sites.
